(19) United States
(12) Patent Application Publication
(10) Pub. No.: US 2009/0053302 A1
(43) Pub. Date: Feb. 26, 2009

(54) CANCER TREATMENTS

(76) Inventor: Parthenios Boulikas, Attiki (GR)

Correspondence Address: BROOKS KUSHMAN P.C., 1000 TOWN CENTER, TWENTY-SECOND FLOOR, SOUTHFIELD, MI, 48075, US

(21) Appl. No.: 12/281,541

(22) PCT Filed: Mar. 5, 2007
(86) PCT No.: PCT/GR2007/000015

§ 371 Date: Sep. 3, 2008

(30) Foreign Application Priority Data
Mar. 3, 2006 (GR) 20060100144

(51) Int. Cl. A61K 9/127; A61K 31/282; A61K 33/24; A61P 35/00

(52) U.S. Cl. 424/450; 514/492; 424/649

(57) ABSTRACT

The present invention relates to liposome comprising encapsulated oxaliplatin and methods for making encapsulated oxaliplatin. The invention also relates to liposomes comprising oxaliplatin and another anticancer drug. The liposomes of the invention are useful in cancer treatments.

### FIELD OF THE INVENTION

The present invention relates to liposome comprising encapsulated oxaliplatin and methods for making encapsulated oxaliplatin. The oxaliplatin liposome can be used for killing cancer cells in a variety of human and animal malignancies. The invention also relates to liposomes comprising oxaliplatin and another anticancer drug.

### BACKGROUND

Immunotherapy, vaccines, angiogenesis inhibitors, telomerase inhibitors, apoptosis inducers, signal transduction therapies, gene therapy and a number of targeted therapies for cancer are promising arsenals in the fight against cancer but have not demonstrated their virtues in a clinical setting. Cancer research undergoes extensive investments; yet, the five-year relative survival from the four main cancers (breast, lung, colorectal and prostate) have not changed much in the last 25 years. Tumour heterogeneity within the same individual is partly responsible for the failure of targeted therapies (Miklos, 2005). Therefore, classical chemotherapy and hormonal therapies (for breast and prostate cancers) along with radiation and surgical intervention remain the mainstay treatments for the vast majority of cancer patients.

However, improvement of delivery and tumour targeting of preexisting chemotherapy drugs with nanotechnology provides alternative treatment. Oxaliplatin is an antineoplastic agent with the molecular formula C₈H₁₄N₂O₄Pt and the chemical name of cis-[(1R,2 R)-1,2-cyclohexanediamine-N,N][oxalato(2-)-O,O] platinum. Its chemical structure is shown below.

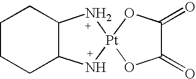

### The Structure of Oxaliplatin

The use of oxaliplatin in cancer therapy has advanced the management of cancer, in particular colorectal cancer. The success of oxaliplatin lies in its ability to induce DNA damage, resulting in bulky adducts as well as intra- and inter-strand crosslinks (Takahara et al, 1995), but also in its ability to induce apoptosis (Boulikas and Vougiouka, 2003). The platinum atom of oxaliplatin forms 1,2-intrastrand crosslinks between two adjacent guanosine residues bending the double helix by approximately 30 degrees toward the major groove. Oxaliplatin has a non-hydrolyzable diaminocyclohexane (DACH) carrier ligand that is maintained in the final cytotoxic metabolites of the drug. Its reaction with DNA and other macromolecules proceeds by hydrolysis of one or both carboxylester groups of oxalate leaving a DACH platinum monoadduct or a bifuctional DACH-platinum crosslink. The intrinsic chemical and steric characteristics of the DACH-platinum adducts appear to contribute to the lack of cross-resistance with cisplatin (reviewed in Di Francesco et al, 2002). Alkaline hydrolysis of oxaliplatin gives the oxalato monodentate complex (pKa 7.23) and the dihydrated oxaliplatin complex in two consecutive steps. The monodentate intermediate is assumed to rapidly react with endogenous compounds (Jerremalm et al, 2003). The crystal structures of oxaliplatin bound to a DNA dodecamer duplex with the sequence 5′-d(CCTCTGGTCTCC) has been reported; the platinum atom forms a 1,2-intrastrand cross-link between two adjacent guanosine residues bending the double helix by approximately 30 degrees towards the major groove. The crystallography provided structural evidence for the importance of chirality in mediating the interaction between oxaliplatin and duplex DNA (Spingler et al, 2001).

However, despite its advantages, the use of oxaliplatin is associated with a unique pattern of side-effects which include neurotoxicity, hematologic toxicity and gastrointestinal tract toxicity. There is a significant risk of grade ¾ neutropenia to patients. Nausea and vomiting is usually mild to moderate. Nephrotoxicity is mild allowing administration of oxaliplatin without hydration. Sometimes, severe side effects may be observed such as tubular necrosis.

Furthermore, cellular resistance to free oxaliplatin has been observed, preventing the potential efficacy of free oxaliplatin. Resistance develops by clonal expansion of a tumor cell that has an advantage and can grow in the presence of oxaliplatin. Several mechanisms have been proposed to explain development of resistance to oxaliplatin in tumors of patients:

1. Resistant cells have developed a mechanism to limit transport of oxaliplatin across their cellular membrane and thus limit the intracellular levels of the drug. This is the most important mechanism for acquisition of resistance to oxaliplatin by tumor cells. The liposomal encapsulation of oxaliplatin described here circumvents this mechanism of resistance to oxaliplatin because of the fusogenic lipid DPPG in the liposomally encapsulated oxaliplatin formulation and because of the nanoparticles size of the drug (average 100 nm) that is avidly phagocytosed by tumor, compared to normal cells.
2. Resistant cells have higher levels of glutathione, metallothioneins or other compounds that detoxify oxaliplatin.
3. Resistant cells have developed a faster repair in DNA lesions after oxaliplatin damage.
4. Other mechanisms for resistance have been proposed that are connected to the signaling of mitochondrial or nuclear apoptotic pathways responsible for the decision of the damaged cell to undergo apoptosis or to repair the damage; it is the decision to repair the damage that will result in the accumulation of mutations at the DNA level that can further change the phenotype of the tumor clone (chromosomal breakpoints resulting in translocations and other chromosomal aberrations).

Therefore, the development of less toxic and more efficient alternatives to the administration of the free drug oxaliplatin is a major challenge. The development of such alternatives could solve several of the problems of cancer therapy.

Liposomes are microscopic vesicles composed of a phospholipid bilayer that are capable of encapsulating active drugs. Liposomal drugs are promising nanovehicles for drug delivery. The liposomally encapsulated cisplatin (sold under the TM Lipoplatin® by Regulon Inc., Mountain View, Calif., U.S. Pat. No. 6,511,676) has been shown to reduce the nephrotoxicity and neurotoxicity of cisplatin, while targeting tumors after systemic delivery in patients.

Oxaliplatin is a drug that has a spectrum of activity, mechanisms of action and resistance different from those of cisplatin. Oxaliplatin adduct lesions are repaired by the nucleotide excision repair system. Oxaliplatin is detoxified by glutathione (GSH)-related enzymes. ERCC1 and XPA expression was predictive of oxaliplatin sensitivity in six colon cell lines in vitro (Arnould et al, 2003). Oxaliplatin has been reported to have better efficacy than cisplatin for colorectal cancers.

Cisplatin and oxaliplatin have substantial structural differences which lead to different side effects during chemotherapy.

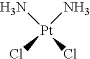

### The Structure of Cisplatin

For example, the side effects of cisplatin are nephrotoxicity, peripheral neuropathy, ototoxicity, and severe gastrointestinal toxicity

(for references see McKeage M J: Comparative adverse effect profiles of platinum drugs. Drug Saf 13: 228-44, 1995, Hanigan M H and Devarajan P: Cisplatin nephrotoxicity: molecular mechanisms. Cancer Ther 1, 47-61, 2003).

There exists a need of reducing the difficulties in the administration of oxaliplatin to reduce the high toxicity of free oxaliplatin when used in therapy, and of targeting tumours and providing efficient treatment to patients with tumours resistant to chemotherapy.

Furthermore, as different drugs appear to have better efficacy in the fight against different cancer cells and in respect of the position and the stage and anatomy of the malignancy, there exists the need to be able and administer in an effective way simultaneously more than one drug or genes in a combination therapy.

The present invention is aimed at solving or at least mitigating these problems by encapsulating oxaliplatin and, in another aspect, oxaliplatin and another anti cancer drug into a liposome. This increases the efficacy of the drug.

### SUMMARY OF THE INVENTION

The present invention provides liposomes comprising encapsulated oxaliplatin and having a different composition of lipids in their outer and inner membrane and methods for making such liposomes. The liposomes comprise a lipid molecule with a negatively charged (anionic) headgroup. The invention also provides liposomes having encapsulated oxaliplatin and another drug and methods for making such liposomes. Further provided are the use of such liposomes in the treatment of cancer.

In a first aspect, the invention relates to a method for forming a micelle comprising oxaliplatin, the method comprising combining an effective amount of oxaliplatin and a negatively charged phosphatidyl glycerol lipid with a solvent.

In a second aspect, the invention relates to a method for encapsulating oxaliplatin into a liposome comprising combining an oxaliplatin micelle according to the invention with a preformed liposome or lipids.

In a third aspect, the invention relates to a method for encapsulating oxaliplatin into a liposome comprising the following steps:

a) forming a micelle comprising oxaliplatin by combining an effective amount of oxaliplatin and a negatively charged phosphatidyl glycerol lipid with solvent and
b) combining said oxaliplatin micelle with a preformed liposome or lipids.

In a fourth aspect, the invention relates to a micelle comprising an effective amount of oxaliplatin and a negatively charged phosphatidyl glycerol lipid.

In a fifth aspect, the invention relates to a liposome comprising an effective amount of oxaliplatin wherein the inner and outer layer of the liposome comprise different lipids. Other aspects of the invention relate to the use of the liposome in the treatment of cancer and a method of treating cancer by administration of the liposome.

In another aspect, the invention relates to a liposome comprising an effective amount of oxaliplatin and another anticancer drug.

In a further aspect, the invention relates to a liposome comprising an effective amount of oxaliplatin and an anticancer gene.

The invention also provides administration schedules for the pharmaceutical formulations, i.e. the liposomes, of the invention.

In a related further aspect, the invention concerns a combination therapy comprising administering an effective amount of gemcitabine and a liposome encapsulating an effective amount of cisplatin. Also provided is the use of a liposome having encapsulated cisplatin in the preparation of a medicament for the treatment of a human patient affected by cancer and a method for treating cancer, by combination therapy involving administration of said liposome and gemcitabine.

### DETAILED DESCRIPTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The invention relates to a method for the encapsulation of oxaliplatin into liposomes having a different lipid composition in their inner than in the outer membrane bilayer.

In a first aspect, the invention relates to a method for forming a micelle comprising oxaliplatin, the method comprising combining an effective amount of oxaliplatin and a negatively charged with a solvent solution. The lipid is characterised in that it comprises a negatively charged (anionic) headgroup. Preferably, the lipid is phosphatidyl glycerol lipid.

Preferably, the solvent is ethanol. However, other solvents known to the skilled person, such as a carbohydrate solvent, may also be used. Methanol may be another suitable solvent.

The term oxaliplatin as used herein refers to oxaliplatin and any oxaliplatin analogues or derivatives. The liposomally encapsulated oxaliplatin of the invention is also referred to herein by its brand name LIPOXAL®.

The term negatively charged phosphatidyl glycerol lipid according to the invention relates to a negatively charged phosphatidyl glycerol lipid or a derivative thereof. These lipids are characterised in that they comprise a negatively charged (anionic) headgroup. Thus, the term is used to describe any lipid having the ability to form micelles and having a net negatively charged head group. The negatively charged phosphatidyl glycerol lipid according to the different aspects of the invention may be selected from dipalmitoyl phosphatidyl glycerol (DPPG), dimyristol phosphatidyl glycerol (DMPG), diaproyl phosphatidyl glycerol (DCPG), distearoyl phosphatidyl glycerol (DSPG) or dioleyl phosphatidyl glycerol (DOPG). In a preferred embodiment, the negatively charged phosphatidyl glycerol lipid is DPPG.

The ethanol solution according to the invention is preferably at 20 to 40%, preferably about 30% ethanol. The molar ratio of oxaliplatin to the negatively charged phosphatidyl glycerol lipid is in a range of 1:1 to 1:2. Preferably, the ratio is 1:1.

Thus, according to one embodiment of the first aspect of the invention, oxaliplatin is mixed with DPPG, at a 1:1 to 1:2 molar ratio in 20-40% ethanol, in the presence of a buffer such as ammonium sulfate (10-200 mM), or Tris buffer (10-100 mM), or sodium Phosphate buffer (10-200 mM) at a pH 6.5-8.0 to achieve about 5 mg/ml final oxaliplatin concentration. The mixture is heated at 30-60 degrees Celsius and incubated for 20 min to 3 h. Under these conditions the positively-charged imino groups on the oxaliplatin molecule are brought with interaction with the negatively-charged groups on the DPPG molecule forming reverse micelles in ethanolic solutions.

In a second aspect, the invention relates to a method for encapsulating oxaliplatin into a liposome comprising combining an oxaliplatin micelle according to the invention with a preformed liposome or lipids.

In a third aspect, the invention relates to a method for encapsulating oxaliplatin into a liposome comprising the following steps:

c) forming a micelle comprising oxaliplatin by combining an effective amount of oxaliplatin and a negatively charged phosphatidyl glycerol lipid with a solvent and
d) combining said oxaliplatin micelle with a preformed liposome or lipids.
In one embodiment of the methods, the micelle is mixed with a preformed liposome.

The preformed liposome or lipids used in the methods of the invention and thus, the liposome of the invention may comprise negatively and/or positively charged lipids, such as phospholipids. Many phospholipids can be used in the present invention. For example, phosphatidylcholines, phosphatidylethanolamines, distearoylphophatidyl-ethanolamine, phosphatidylserines, phosphatidylinositols, lysophosphatidylcholines, phosphatidylglycerols. sphingomyelins or phosphatidic acid all find use in the present invention. Also used can be ceramide or other lipid derivatives. For the purpose of modifying the stability or permeability of the lipid membrane, an additional lipophilic component can be added such as, for example, cholesterol or another steroid, stearylamine, phosphatidic acid, dicetyl phosphate, tocopherol, or lanolin extracts.

The lipids may be selected from but are not limited to DDAB, dimethyldioctadecyl ammonium bromide; DMRIE: N-[1-(2,3-dimyristyloxy)propyl]-N,N-dimethyl-N-(2-hydroxyethyl) ammonium bromide; DMTAP: 1,2-dimyristoyl-3-trimethylammonium propane; DOGS: Dioctadecylamidoglycylspermine; DOTAP: N-(1-(2,3-dioleoyloxy)propyl)-N,N,N-trimethylammonium chloride; DOTMA: N-[1-(2,3-dioleyloxy)propyl]-n,n,n-trimethylammonium chloride; DPTAP: 1,2-dipalmitoyl-3-trimethylammonium propane; DSTAP: 1,2-disteroyl-3-trimethylammonium propane.

In one embodiment of the invention, the oxaliplatin liposomes comprise DPPG, cholesterol and HSPC (hydrogenated soy phospahatidyl choline). Said encapsulation intends to reduce the adverse reactions of the cytotoxic agents without reducing effectiveness.

The liposomal preparation of the invention may also comprise an ammonium salt, such as ammonium chloride, ammonium sulfate or any other ammonium salt.

The negatively charged phosphatidyl glycerol lipids according to the invention, which are used to form the micelle and which are part of the liposome membrane, provide the advantage that they enhance the permeability of the cell membrane for delivery of drug into the cytosol. The liposome can thus fuse with the membrane of the cell and release its contents into the inside of the cell. These properties are termed fusogenic. Thus, because of these fusogenic properties and the phagocytosis mechanism, the liposomal formulations of oxaliplatin according to the invention are capable of passing through the cell membrane of the tumour cell and thus have applications in the treatment of oxaliplatin-resistant or drug-resistant tumours.

According to another embodiment, the complexation into the same liposome of oxaliplatin with negatively-charged phosphatidyl glycerol lipids results in very high (50-100%) encapsulation efficiency, minimizing drug loss during product manufacturing.

The method for encapsulation according to the invention is based on the formation of reverse micelles between oxaliplatin with a negatively-charged lipid molecule as described herein. Reverse micelles are held by electrostatic interaction between the positively-charged amino groups of oxaliplatin and a negatively-charged phosphate groups of the phosphatidyl glycerol lipid, for example DPPG, and direct their hydrophobic chains of the phosphatidyl glycerol lipid toward the ethanolic solution, thus engulfing oxaliplatin molecules. The oxaliplatin-phosphatidyl glycerol lipid reverse micelles are converted into liposomes by mixing them with pre-made liposomes or lipids, this may be followed by dialysis and extrusion through membranes, to remove the ethanol, or dilution with water, extrusion through filters, with or without concentration with high pressure filtration. This results in entrapping and encapsulating oxaliplatin to very high yield. The lipid composition of the liposomes during the preparation method determines to a high extent the lipid composition of the outer surface of the nanoparticle.

In one embodiment of the different aspects of the invention, a coating which enables the liposome of the invention to evade immune surveillance can be added. Preferably, the coating is a polymer. The coating can be added either at the liposome stage or post-insertionally at the formed nanovehicle. Thus, the liposomes of the invention may comprise such coating. Polymers that can be used according to the invention include polyethylene glycol (PEG), polymethylethylene glycol, polyhydroxypropylene glycol, polypropylene glycol, polymethylpropylene glycol, polyhydroxypropylene oxide, polyoxyalkylenes, polyetheramines. Additional polymers include polyvinylpyrrolidone, polyvinylmethylether, polymethyloxazoline, polyethyloxazoline, polyhydroxypropyloxazoline, polyhydroxypropylmethacry-lamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide, hyaluronic acid. A preferred polymer is PEG. For example, distearoylphosphatidylethanolamine may be derivatised with PEG to lead to PEG-derivatized distearoylphosphatidylethanolamine (PEG-DSPE). The polymers may be employed as homopolymers or as block or random copolymers.

The liposomal oxaliplatin nanovehicles disclosed in the present invention can evade immune surveillance because of polymer coating, can circulate for extended periods in body fluids, can redistribute from tissue pools into tumors and can concentrate preferentially into solid tumors and metastases after intravenous injection to animals and humans by extravasation through the compromised vasculature that has imperfections in its endothelium during the process of neoangiogenesis.

An advantage of the encapsulation method described in the present invention is that the drug in the liposome nanovehicle will reach primary tumors and metastases by preferential extravasation through the leaky tumor vasculature and thus have an enhanced anticancer activity. The fusogenic lipid DPPG enhances the fusion of the nanoparticles with the tumor cell membrane whereas a higher uptake of the liposomal oxaliplatin is also enhanced by the avidity of tumor cells for phagocytosis.

Furthermore, a ligand may be conjugated to the polymer coating of the liposomes of the invention.

For example, the ligand may be a peptide, for example an antibody. Peptides may be inserted postinsertionally, for example as Peptide-PEG-DSPE conjugates. Peptides according to the invention include, but are not limited to those that are derived from the endostatin, antithrombin, anastellin, angiostatin, PEX, pigment epithelial-derived factor, thrombospondin (TSP)-1 and -2 primary structures and those that are able to exert a dual anticancer activity: that of restricting tumor angiogenesis via, for example, a 27-amino-acid peptide corresponding to the NH₂-terminal domain of endostatin attached to PEG-DSPE (FIG. 17) and also exerting antitumor activity from the oxaliplatin molecules encapsulated into the same antiangiogenesis peptide-carrying liposome.

A preferred peptide is endostatin. Endostatin, the 20-kDa C-terminal proteolytic fragment of the noncollagenous domain 1 (NC1) of the basement membrane protein collagen XVIII, inhibits cell proliferation and migration and is an endogenous inhibitor of tumor angiogenesis and tumor growth. A major problem in reconciling the many reported in vitro effects of endostatin is the lack of a high-affinity receptor. Chronic exposure to endostatin blocks endothelial cell proliferation, and migration and induces endothelial cell apoptosis thereby inhibiting angiogenesis; endostatin stimulated acute phosphorylation of endothelial nitric oxide synthase (eNOS) at Ser116, Ser617, Ser635, and Ser1179, and dephosphorylation at Thr497 in cultured bovine aortic endothelial cells, events associated with eNOS activation. Indeed, nitric oxide (NO) is promoting angiogenesis. Short-term exposure of endothelial cell to endostatin, therefore, unlike long-term exposure which is anti-angiogenic, may be pro-angiogenic (Li et al, 2005). A 27-amino-acid peptide corresponding to the NH2-terminal domain of endostatin elicited its full antiangiogenic activity and had strong antitumor activity; three histidines that are responsible for zinc binding were essential for the anticancer properties of the peptide (Tjin Tham Sjin et al, 2005, Tjin Tham Sjin R M, Satchi-Fainaro R, Birsner A E, Ramanujam V M, Folkman J, Javaherian K. A 27-amino-acid synthetic peptide corresponding to the NH2-terminal zinc-binding domain of endostatin is responsible for its antitumor activity (Cancer Res. 2005 May 1; 65(9):3656-63. Li C, Harris M B, Venema V J, Venema R C. Endostatin induces acute endothelial nitric oxide and prostacyclin release. Biochem Biophys Res Commun. 2005 Apr. 15; 329(3):873-8.)

Peptide ligands are derived easily to those skilled in the prior art by selection of peptide libraries for ligands able to interact specifically with peptide epitopes derived from tumor-specific antigens overexpressed at the surface of the tumour cell. Attachment of these peptides at the end of PEG with the chemistry shown in FIG. 17 gives oxaliplatin encapsulating liposomes able to be directed to specific tumours. Table 1 diagrammatically depicts tumour antigens from which peptides exposed to the external cell surface can be derived, synthesized, and used to derive peptide ligands from random peptide libraries with high affinity for the tumour antigen. Such peptide ligands are then covalently attached to the lipid-polymer molecule, for example a PEG-DSPE molecule, that is inserted at the liposome particle.

Other ligands may be selected from the group consisting of transferrin, folic acid, hyaluronic acid, a sugar chain such as galactose or mannose, a monoclonal antibody, pyridoxal phosphate, vitamin B12, sialyl Lewis X, epidermal growth factor, basic fibroblast growth factor, vascular endothelial growth factor, vascular cell adhesion molecule (VCAM-1), intercellular adhesion molecule (ICAM-1), platelet endothelial adhesion molecule (PECAM-1), an Arg-Gly-Asp (RGD) peptide, or an Asp-Gly-Arg (NGR) peptide, and a Fab′ fragment of a monoclonal antibody.

In one embodiment, the liposomal oxaliplatin particles are modified on their surface with PEG-DSPE-folate conjugates inserted after formation of the liposome particle to direct the particles to tumors overexpressing folate receptors.

TABLE 1
Ligands

| Molecule | Disease indication | Reference |
|---|---|---|
| EGFR | NSCLC, breast cancer, bladder, | Yarden Y. & Sliwkowski M. X. |
| | ovarian cancer | (2001), Lynch et al., (2004) |
| HER/NEU | Breast, ovarian, lung cancer, ovarian, | Koeppen H. K., et al., (2001), |
| | colorectal, prostate cancer | Slamon D. J., et al., (1989) |
| VGEFR | Angiogenesis, NSCLC | Cardones A. R. & Banez L. L. (2006), |
| | | Rosen L. S. (2005) |
| FR (Folate Receptor) | Ovarian, breast, brain, lung, | Sudimack J. and Lee R. J. (2000), |
| | colorectal cancer | Garin-Chesa P. et al., (1993), Ross J. F., |
| | | et al., (1994) |
| MUC | Breast, lung, colorectal, prostate, | Liu., et al., (2004), Finn O. J. et al., |
| | kidney, pancreatic cancer | (1995) |
| Hsp90 (Molecular | Breast, lung cancer | Whitesell L. & Linquist S. L. |
| chaperone) | (involved in the chaperoning of | (2005), Yu X. et al., (2002) |
| | many cancer antigens) | |
| CD20 | Non-Hodgkin's lymphoma, | Perosa F., et al., (2005), |
| | autoimmune diseases | Wojciechowski W., et al., (2005) |
| CEA | Colorectal cancer | Liu K., et al., (2004) |
| TAA (Tumor-associated | breast cancer, NSCLC, ovarian, | Bandic D., et al., (2006), Ito S., et al., |
| antigens: MAGE) | gastric cancer | (2005) |
| EpCAM | breast, ovarian, colon cancer | Osta W. A., et al., (2004) |

Peptides directed against tumor antigens can also be added at the end of a polymer, for example a PEG-polymer for multifunctionalization giving to the nanoparticles the property to target specific tumors overexpressing specific surface antigens.

In one embodiment, the liposomal oxaliplatin particles are also modified with folic acid directing the oxaliplatin lipo-nano-particles to ovarian (and other) malignant cells overexpressing folate receptors.

In another embodiment, the liposomal oxaliplatin particles are also modified with Her2/neu ligands directing the oxaliplatin nano-particles to breast cancer cells overexpressing Her2/neu.

The liposomal formulations of oxaliplatin according to the invention circumvent the problem of resistance to free oxaliplatin caused by reduced uptake of the drug in resistant tumors. Thus, the formulations have applications in the treatment of oxaliplatin-resistant tumors. The liposomal formulations of oxaliplatin according to the invention also display a lower toxicity profile than the free drug oxaliplatin (free oxaliplatin) in human clinical trials against a variety of solid malignancies. Further, because the spectrum of side effects of these liposomal formulations of oxaliplatin are different than those of free oxaliplatin and the mechanism of entry into tumor cells is also different, the liposomal formulations of oxaliplatin according to the invention may have advantages clinical applications in non-small cell lung cancer, in breast cancer, in ovarian cancer, in head and neck cancer, in metastatic prostate cancer and in several other solid tumours, in addition to colorectal and gastric cancers.

In one embodiment, the liposomally encapsulated oxaliplatin of the invention is able to lower the levels of bilirubin (FIG. 2) or the bone metastases (FIG. 3) in treated patients.

In another embodiment, the liposomal preparations described herein can be used after intravenous infusion to lower the side effects of oxaliplatin, especially gastrointestinal toxicity and of the other co-encapsulated drugs.

The liposomal preparations according to the invention can be directed preferentially to human tumours and their metastases.

Thus, in a further aspect, the invention relates to a liposome comprising oxaliplatin as described herein for use as a medicament.

In another aspect the invention relates to the use of a liposome having encapsulated oxaliplatin in the manufacture of a medicament for the treatment of cancer.

The invention also relates to a method of treatment of cancer comprising administering a liposome having encapsulated oxaliplatin according to the invention to a patient.

Different types of cancer may be treated, including colorectal cancer, gastric cancer, pancreatic, breast cancer, non-small cell lung cancer, in ovarian cancer, head and neck cancer, prostate cancer, testicular, intestinal cancer, oesophageal or urothelial cancer. Preferably, the treatment is for colorectal, gastric or pancreatic cancer.

The liposome is administered weekly by intravenous infusion at a dosage of 100 to 350 mg/m². Preferably, administration is at a dosage of 300 mg/m², but other possible dosages are 100 mg/m², 150 mg/m², 200 mg/m² or 250 mg/m². In one embodiment, the administration is in 2 to 5 cycles. Each cycle is 8 to 12 weeks and is followed by a one or two weeks rest. Preferably, the intravenous weekly infusion is for 3 hours.

In another embodiment, administration is very two weeks.

These administration schedules described above may also be used when oxaliplatin is administered as a combination therapy as described herein.

In another aspect, the invention relates to a method for making micelles and/or liposomes comprising two anticancer drugs, oxaliplatin and another drug. The method is as described herein with reference to making oxaliplatin liposomes but includes the step of including another anticancer drug in the micelle or liposome.

Thus, in a further aspect, the invention relates to liposomes comprising encapsulated oxaliplatin and another anticancer drug. The drugs are thus encapsulated within the same liposome. This has the advantage that they can be delivered together to the target. It is also possible and within the scope of the invention to include more than one other anticancer drug in the liposome.

In one embodiment, at least two anticancer drugs with different mechanisms of action are included in the same liposome according to the invention. Therefore, the tumour cell can be targeted with two independent mechanisms, leading to a better clinical success.

The other anticancer drug can be selected from compounds such as platinum compounds (such as cisplatin, carboplatin), antimetabolite drugs (such as 5-fluorouracil, cytarabine, gemcitabine, pentostatin and methotrexate), anthracycline drug which targets DNA (such as doxorubicin and epirubicin), drugs which target DNA or drugs which target topoisomerases or other chemotherapy drugs.

In a preferred embodiment, the other drug is selected from cisplatin, docetaxel, paclitaxel, gemcitabine, navelbine, doxorubicin, irrinotecan, SN-38, gemcitabine or 5-fluorodeoxyuridine.

By including the two drugs in the same liposome, it is possible to use a lower dose of each drug than when each drug is administered alone. The two drugs may act in a synergistic manner, thus incurring more damage to the tumour cell with lower side effects.

In another preferred embodiment, cisplatin and oxaliplatin are coencapsulated into the same liposome nanoparticle. Thus, the same tumor cell can be attached simultaneously by both, cisplatin and oxaliplatin. The side effects of cisplatin (nephrotoxicity, neurotoxicity, nausea/vomiting) are different from the side effects of liposomal cisplatin (hematological toxicity). The side effects of oxaliplatin are also different from the side effects of liposomal oxaliplatin (neuropathy). Thus, the same tumor cell can be targeted with at least two independent mechanisms, while otherwise (if not administered encapsulated in the same liposome) the two drugs (oxaliplatin and cisplatin) would most probably each target a different cell. Besides, the administration of a combination of different drugs encapsulated in the same liposome makes it possible to use lower dosages for attaining efficacy thus avoiding or reducing the toxicity of the drugs. More particularly, by lowering the dosage of oxaliplatin the inventors have found that the side effect of neurotoxicity may be limited whilst by lowering the dosage of cisplatin the side-effect of myelotoxicity may be limited. As a result, there is improvement of the profile of neurotoxicity and myelotoxicity of the administered liposomal oxaliplatin and liposomal cisplatin, respectively, whereas at the same time it is possible to incur the same or a higher damage to the tumours after systemic administration. Thus, the combination of cisplatin and oxaliplatin into the same liposome allows the administration of each of the said drugs at lower doses, under conditions where the side effects of the liposomal drugs are even more minimized.

In another embodiment, the liposomally encapsulated oxaliplatin of the invention is combined with the drug doxorubicin (DOX) which is encapsulated into the same liposomal oxaliplatin particle as oxaliplatin as described in the methods of the invention. Surprisingly, the inventors have found that this may lower the dose of oxaliplatin and by consequence the neurotoxicity of the administered liposomal oxaliplatin whilst also reducing the dose of DOX. This reduces the cardiotoxicity and other side effects of DOX whilst inflicting the same or a higher damage to the tumours.

In another embodiment, the liposome comprises oxaliplatin and 5-fluorouracil. Oxaliplatin in combination with 5-fluorouracil has been recently approved for the treatment of metastatic colorectal cancer. However, there are serious problems in the administration of such drugs, mainly due to the important side effects of either drugs, which are minimized with their liposomal encapsulation as described in the invention. Furthermore, by combining the drugs as described herein, effectiveness of the treatment is increased.

The invention also relates to the encapsulation of oxaliplatin and an anticancer gene in the same liposome. Thus, liposomes according to the invention may comprise oxaliplatin and an anticancer gene. The anticancer genes used include, but are not limited to p53, IL-2, IL-12, angiostatin, and oncostatin.

In another aspect, the invention relates to a combination therapy wherein oxaliplatin is administered together with another drug or gene as specified herein wherein both drugs are encapsulated in the same liposome. Thus, the liposomes comprising oxaliplatin and another anticancer drug or gene can be used in the manufacture of a medicament for the treatment of cancer or in a method of treating cancer. Furthermore, the invention relates to a first medical use of the combination liposomes.

A skilled person will appreciate that administration schedules and dosage of the components vary according to the other drug present. As for oxaliplatin, a dosage and dosage range as described herein can be used. Furthermore, the administration schedule of the combination liposome may be as described herein for oxaliplatin.

In one embodiment, the liposomally encapsulated oxaliplatin of the invention is adminstered to cancer patients 150-300 mg/m² weekly (Days 1, 8, 15) for 12 weeks as monotherapy or in combination with 1 g/m² gemcitabine on days 1, 8 in a 21-day cycle or in combination with docetaxel, paclitaxel, irrinotecan.

In a related aspect, the invention is directed to liposomally encapsulated cisplatin wherein cisplatin is encapsulated in combination with another anticancer drugs as defined herein. Cisplatin can thus be combined in the same liposome particle with any one of the anticancer drugs of paclitaxel, docetaxel, irrinotecan, SN-38, gemcitabine, 5-fluorodeoxyuridine. The advantage is that the same tumor cell is being attacked simultaneously by cisplatin and one other drug, thus, achieving a more effective killing because of the two independent molecular mechanisms involved. For example, cisplatin will elicit mitochondrial and nuclear signalling for apoptosis as well as DNA crosslinks arresting replication whereas docetaxel will act at the tubulin polymerization.

Advantageously, liposomally encapsulated cisplatin is encapsulated into the same liposome in combination with gemcitabine, using the methods as described herein.

In another aspect, the oxaliplating comprising liposome of the invention may be administered together with another anticancer drug, but the other drug doe snto form part of the same liposome. The other drug is as described herein and is preferably selected from cisplatin, docetaxel, paclitaxel, gemcitabine, navelbine, doxorubicin, irrinotecan, SN-38, gemcitabine or 5-fluorodeoxyuridine.

Furthermore, in a separate aspect, the invention relates to the administration of Lipoplatin® in combination with gemcitabine. Thus, a combination therapy of Lipoplatin® and gemcitabine is an object of the invention. Also provided is the use of Lipoplatin® in the preparation of a medicament for the treatment of a human patient affected by cancer, by combination therapy involving administration of Lipoplatin® and another drug that is not encapsulated in the same liposome.

The other drug can be administered at the same time as Lipoplatin® or at a different time.

Preferably, the other drug is gemcitabine and the administration leads to clinical improvement. Preferably, the cancer treated is pancreatic cancer, but other cancers, such as colorectal cancer, gastric cancer, breast cancer, non-small cell lung cancer, ovarian cancer, head and neck cancer, prostate cancer, testicular, intestinal cancer, bladder, esophageal or urothelial cancer, may also be treated. The dosage used for gemcitabine is 800 to 1000 mg/m², preferably 1000 mg/m². The lipoplatin dose is 100 to 125 mg/m², preferably 100 mg/m².

Administration of Lipoplatin® and gemcitabine is intravenous. Lipoplatin® is preferably administered as an 8 hour IV infusion every two weeks (day 1 and day 15). Gemcitabine is preferably administered as a 60 min iv infusion every two weeks. Administration of the compounds may be in cycles of 4 weeks.

The invention is further illustrated with reference to the following figures and examples. The examples show that the administration of oxaliplatin liposomes leads to clinical improvement, i.e. has a clinical effect in the treatment of cancer. Example II shows, that administration of Lipoplatin® and gemcitabine provides clinical benefits, thus leading to clinical improvement.

### DESCRIPTION OF THE FIGURES

FIG. 1: Schematic representation of the liposomal oxaliplatin shown as yellow rectangles. Lipid molecules are depicted with spherical hydrophilic heads. Red random chains on the surface of the particle represent the PEG molecules that give to the particle its ability to escape destruction from macrophages in the liver, opsonization (interaction with serum proteins and other macromolecules) in the blood and the ability to extravasate into solid tumors and metastases after systemic delivery (also its small size of 100 nm).

FIG. 2: Reduction in bilirubin levels in a patient (TK) with colorectal cancer and liver metastases. The patient was going into hepatic come from the very high levels of bilirubin in the blood (50 mg/100 ml). Injection of Liposomal oxaliplatin at doses of 200 mg/m² on day 1, day 8, day 15 and day 22 resulted in progressive reduction of total bilirubin from 50 to 12 mg/m². Most likely this resulted from reduction in the liver metastases that occluded the biliary tract. Further treatments on days 31 and 37 did not stop the disease progression as deduced from bilirubin levels.

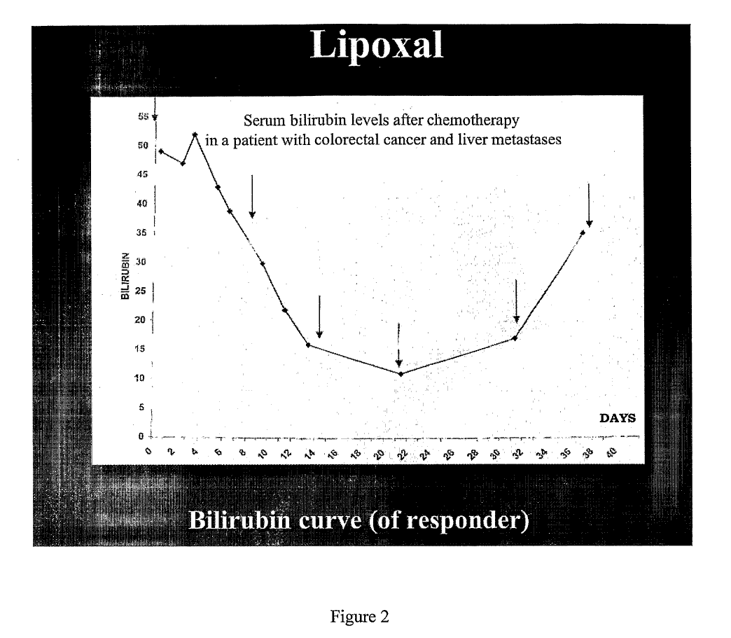

FIG. 3: Reduction in bone metastases after monotherapy with liposomal oxaliplatin. A patient (EK) suffering with gastric cancer and bone metastases was treated with 150 mg/m² every 7 days for 10 weeks. There was a significant improvement in quality of life, much less pain, lesser use of analgesics and the patients was able to perform his work on a daily basis.

FIG. 4: Coencapsulation of cisplatin and oxaliplatin into the same liposome particle and further postinsertional modification of the particles with peptide-PEG-LIPID conjugates to direct these to specific cell types with surface receptors recognized by the peptides or ligands. The scheme also depicts peptide chains (red color) added at the end of PEG molecules for the multifunctionalization of the liposome particles and their preferential direction to specific tumors. In this case, specific tumor antigens are recognized by the peptide moiety on the surface of the liposome. For example, epidermal growth factor) peptide epitopes able to bind to the part of the EGFR exposed to the outer surface directs said liposomes to tumors overexpressing EGFR.

FIG. 5A: shows maxima levels of ˜14 mg total platinum/ml plasma after liposomally encapsulated oxaliplatin compared to ˜8 mg total platinum/ml plasma after oxaliplatin and these were reached at 20 min for liposomally encapsulated oxaliplatin and at 10 min for oxaliplatin.

FIG. 5B: shows that total platinum levels in rat plasma reached zero at ˜100 h post-injection for free oxaliplatin.

FIG. 6A: shows the total platinum levels in rat plasma in animals treated also with Lipoplatin®.

FIG. 7A: shows the total Platinum levels in kidney tissue in animals treated for 5 hrs and

FIG. 7B shows the same treated for 190 hrs.

FIG. 8A: shows the total Platinum levels in liver tissue in animals treated for 5 hrs and

FIG. 8B shows the same treated for 190 hrs.

FIGS. 9A and 9B: show the total Platinum levels in spleen tissue in animals treated for 190 hrs.

FIG. 10A: shows the total Platinum distribution in rat tissue in animals treated with both free oxaliplatin and liposomally encapsulated oxaliplatin for 5 hrs and FIG. 10B FIGS. 11A and 11B are charts of rats treated repeatedly (11 times) with liposomally encapsulated oxaliplatin.

FIG. 12 is a chart of rats treated repeatedly (6 times) with liposomally encapsulated oxaliplatin.

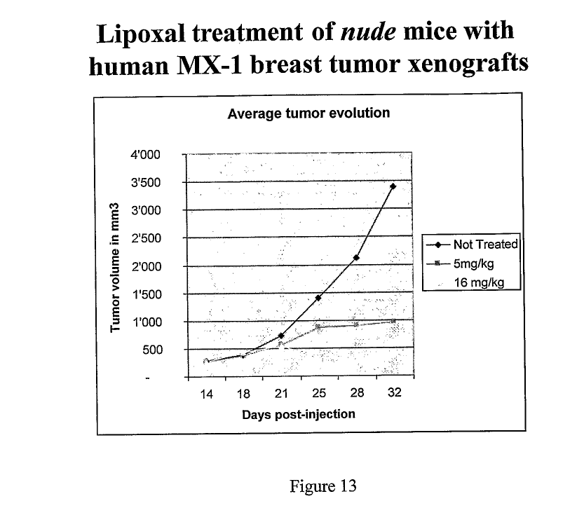

FIG. 13: Lipoxal can induce complete disappearance of human breast cancers in mice after 6 intravenous injections with 4 days intervals at doses of 16 mg/Kg. Oxaliplatin at its MTD (Maximum tolerated dose) can only cause shrinkage, not disappearance of human breast tumors in mice.

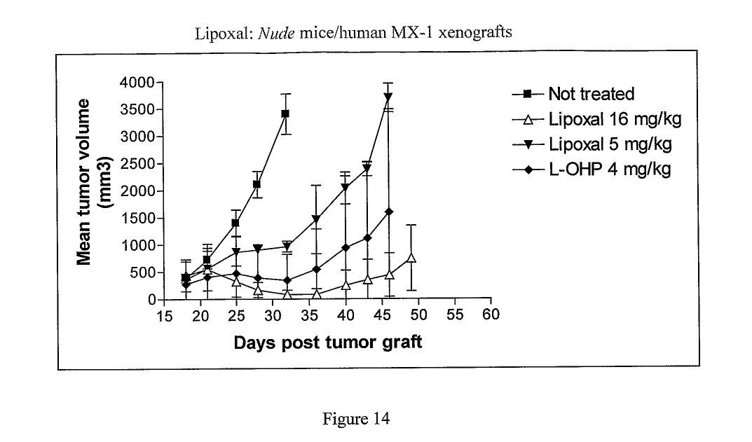

FIG. 14: The dose of 16 mg/Kg liposomal oxaliplatin (Lipoxal) is the most effective in eradicating breast cancer in mouse xenografts. Oxaliplatin at its maximum tolerated dose of 4 mg/Kg has a lower anticancer efficacy in this mouse model followed by a dose of 5 mg/Kg Lipoxal.

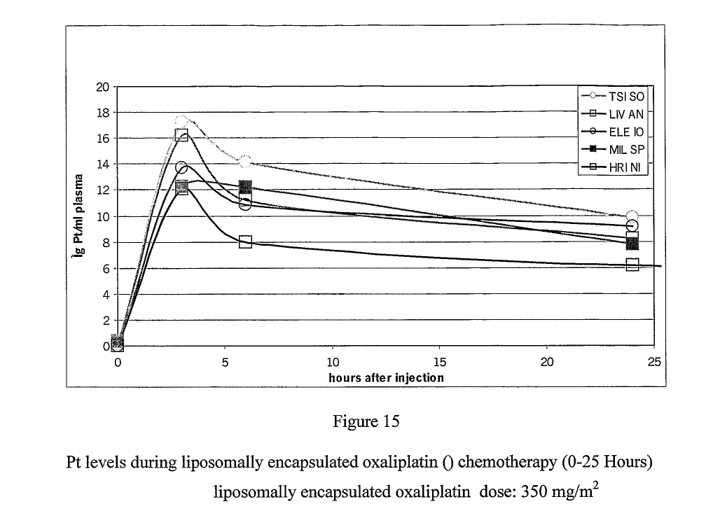

FIGS. 15 and 16 show the results of the clinical trials of liposomally encapsulated oxaliplatin.

FIG. 17: Chemical procedure for coupling peptides to PEG-DSPE.

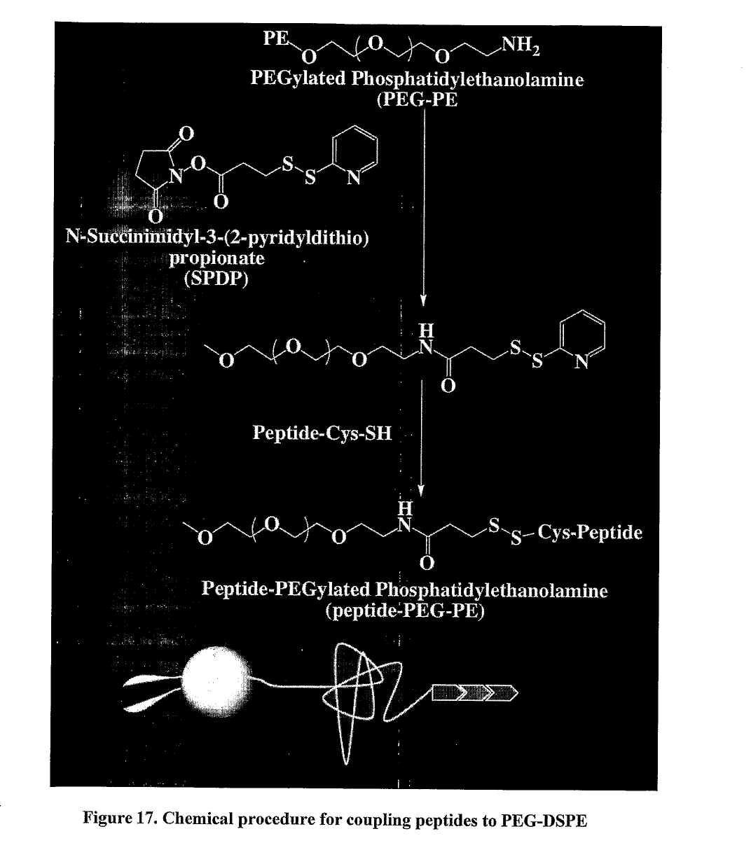

### EXAMPLES

### Example I

### Making Liposomes

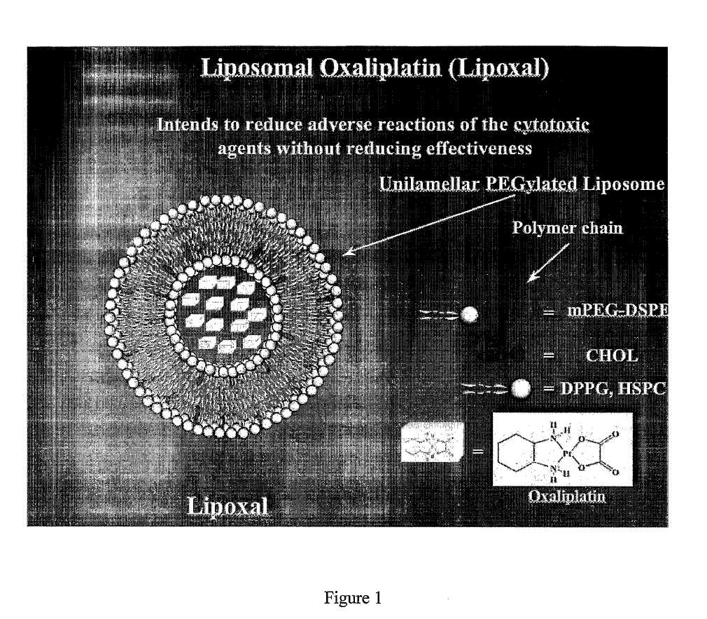

Oxaliplatin is mixed with DPPG (dipalmitoyl phosphatidyl glycerol) or other negatively-charged lipid molecules at a 1:1 molar ratio in 30% ethanol, 0.1 M Tris HCl, pH 7.5 at 5 mg/ml final oxaliplatin in the presence of ethanol solutions at a concentration of 20-40% and under temperature conditions of 30-60 degrees Celsius in the presence of ammonium sulfate (10-200 mM), or Tris buffer (10-100 mM), or sodium Phosphate buffer (10-200 mM) at a pH 6.5-8.0 is incubated for 20 min-3 h. Under these conditions the positively-charged imino groups on the oxaliplatin molecule are brought with interaction with the negatively-charged groups on the DPPG molecule forming in ethanolic solutions reverse micelles (see also the Lipoplatin U.S. Pat. No. 6,511,676). The resulting reverse micelles of oxaliplatin-DPPG are than converted into liposomes encapsulating the oxaliplatin-DPPG monolayer by rapid mixing with preformed liposomes composed of cholesterol, phosphatidyl choline, mPEG-DSPE (polyethylene glycol-distearoyl phosphatidyl ethanolamine), followed by dialysis against saline and extrusion through membranes to downsize the particles to 80-120 nm in diameter. It is the lipid composition of added liposomes that determines the composition of the outer surface of the final oxaliplatin formulation (FIG. 1).

### Example II

### A. Preliminary Clinical Experience with Liposomally Encapsulated Oxaliplatin

### I.A. Animal Studies

The animal studies carried from May 2003 till December 2004 in USA, France, Switzerland and Hellas (Pasteur Institute, Athens) on mouse xenografts by independent laboratories have shown a better therapeutic efficacy of the liposomally encapsulated oxaliplatin compared to mere oxaliplatin as well as a lower toxicity profile and was shown to be better tolerated in mice and rats compared to the free drug oxaliplatin. Furthermore, liposomally encapsulated oxaliplatin could induce complete disappearance or shrinkage of a variety of human cancers in mice after 6-8 intravenous injections in a more effective and less toxic treatment than oxaliplatin.

Liposomally encapsulated oxaliplatin has shown to induce complete disappearance of human breast cancers in mice after 6 intravenous injections with 4 days intervals at doses of 16 mg/Kg. On the other hand the free drug oxaliplatin at its MTD (Maximum tolerated dose) can only cause shrinkage, not disappearance of tumours.

Mice injected with 5 mg/Kg free oxaliplatin died of toxicity and the dose was lowered to 4 mg/Kg. The dose of liposomally encapsulated oxaliplatin was 16 mg/Kg i.v. and the toxicity was lower than 4 mg/Kg free oxaliplatin. The anticancer efficacy of 4 mg/Kg free oxaliplatin was lower than that of 16 mg/Kg liposomally encapsulated oxaliplatin in animals with human tumours.

In the said study animal studies of a liposomally encapsulated oxaliplatin was reported. Intraperitoneal (i.p.) injection of liposomally encapsulated oxaliplatin, or free oxaliplatin as a control, to rats was used to study tissue biodistribution from 10 minutes to 7 days postinjection. Maximum levels of total platinum (Pt) in plasma at a dose of 15 mg/Kg were 14.0 mg/ml plasma after liposomally encapsulated oxaliplatin injection compared to 7.5 mg/ml plasma after free oxaliplatin treatment; these levels were attained at 10-15 min from injection. A similar to plasma pharmacokinetic behavior was observed for kidney tissue; plasma and kidney had the highest levels of platinum among all tissues examined during the first 20 min from injection. Spleen tissue exhibited over 2 times higher levels of platinum after free oxaliplatin treatment compared to liposomally encapsulated oxaliplatin at the same dose level during an extended period of 40-190 h post-injection. Following 11 repetitive administrations of liposomally encapsulated oxaliplatin to rats, spleen attained astonishingly high levels of total Pt among all tissues examined (80 mg/g tissue). Liver exhibited similar pharmacokinetics of Pt accumulation as a function of time after free oxaliplatin versus liposomally encapsulated oxaliplatin treatment. Lipoplatin® for comparison, exhibited similar pharmacokinetic behavior to liposomally encapsulated oxaliplatin in rat kidney from 10 minutes to 7 days but liver pharmacokinetics were similar between the two drugs up to 4 h and there was a higher accumulation of liposomally encapsulated oxaliplatin compared to Lipoplatin® over periods of 7 days. Full biochemical and blood cell counts in rats have established that liposomally encapsulated oxaliplatin exhibited a lower myelotoxicity compared to free oxaliplatin. SGOT transaminase, alkaline phosphatase, bilirubin, creatinine, blood urea, and blood uric acid levels were normal consistent with no hepatic or nephrotoxicity from liposomally encapsulated oxaliplatin in rats. The data show a more extended retention of liposomally encapsulated oxaliplatin in rat tissues consistent with its liposomal PEGylated formulation and a lower toxicity profile.

Injections of Rats with Liposomally Encapsulated Oxaliplatin for Pharmacokinetic Studies

For pharmacokinetic studies 20 Wistar female rats, 2-3 months of age were used of an average body weight of 150 g. Rats were injected in the intraperitoneal cavity with a suspension of 3 mg/ml liposomally encapsulated oxaliplatin giving a final dose of 15 mg/Kg. Two animals per time point were used. Rats were sacrificed at ˜7 min, 20 min, 1.5 h, 3.75 h, 24 h, 40 h, 90 h and 170-180 h postinjection. Blood was collected in heparinized Eppendorf tubes and was centrifuged. Total platinum levels in plasma was determined using furnace Atomic Absorption (AA700 Perkin Elmer).

Repeated Injections of Rats with Liposomally Encapsulated Oxaliplatin for Histology Studies

We were interested determining the damage to various tissues after repeated injection of liposomally encapsulated oxaliplatin to its maximum tolerated dose in rats.

Biochemical and Hematological Analysis in Rats for Toxicity from Liposomally Encapsulated Oxaliplatin

Rats were injected in the intraperitoneal cavity with a suspension of 3 mg/ml liposomally encapsulated oxaliplatin giving a final dose of 15 or 30 mg/Kg. Blood from rats used for plasma pharmacokinetic studies was also analyzed (7 days postinjection) for bone marrow, renal, hepatic and gastrointestinal functions by an independent microbiology laboratory. The parameters examined were hemoglobin, hematocrit, leukocytes, granulocytes, platelets, SGOT transaminase, SGPT transaminase, alkaline phosphatase, total bilirubin, urea, uric acid and creatinine.

### Results

### Toxicology of Liposomally Encapsulated Oxaliplatin in Rats

Rats were injected to a final dose of 15 or 30 mg/Kg with free Oxaliplatin or liposomally encapsulated oxaliplatin. The 30 mg/Kg oxaliplatin group severely lost appetite and exhibited severe weight loss; there was a 33% weight loss in the 30 mg/Kg oxaliplatin group at 7 days post-treatment; the average weight of all animals dropped from 150 g to an average of 100 g after 7 days. On the contrary, animals injected with the same dose of 30 mg/Kg liposomally encapsulated oxaliplatin, showed only a 10% reduction in weight (from an average of 150 g to a the final of 135 g on day 7).

At 7 days postinjection blood drawn into heparinized or non-heparinized tubes from the 15 mg/Kg treated animals and was given to an independent clinical laboratory for full biochemical and hematological analysis. Two animals per group were used. Table 1 shows the average of two measurements. The 15 mg/Kg oxaliplatin group displays a drop in leukocytes to 800,000/mm3 (Grade 4 toxicity according to WHO) compared to 3,400,000/mm3 (Grade 1 toxicity) for the group treated with liposomally encapsulated oxaliplatin. Therefore, liposomally encapsulated oxaliplatin did not cause the extensive reduction in leukocyte counts as compared to free oxaliplatin. Platelet levels were also reduced to a higher extend by oxaliplatin compared to liposomally encapsulated oxaliplatin. The hemoglobin levels were close to normal for both treatments. Therefore, the myelotoxicity of either drug appears to be directed more to the leukocyte and platelet rather than erythropoiesis programs. The SGOT transaminase was elevated by either drug consistent with Grade 2 hepatic toxicity; however, the levels of SGPT transaminase and alkaline phosphatase were not affected; bilirubin, blood urea and creatinine levels were not affected (although blood uric acid levels dropped) consistent with absence of nephrotoxicity caused by either free oxaliplatin or liposomally encapsulated oxaliplatin in rats after i.p. administration.

TABLE 2
Changes in bone marrow, hepatic and kidney functions in rats after
i.p. injection of liposomally encapsulated oxaliplatin or free oxaliplatin.

| | | liposomally | |
|---|---|---|---|
| | | encapsulated | |
| | Oxaliplatin | oxaliplatin | |
| | Rats i.p. | Rats i.p. | |
| | 15 mg/Kg | 15 mg/Kg | |
| | 7 days post | 7 days post | Control |
| | injection | injrction | rats |
| Hemoglobin (gr/dl) | 15.8 | 19 | 16.9 |
| Hematocrit HCT (%) | 42 | 51.5 | 45 |
| Leukocytes (1,000/mm³) | 800 (Grade 4) | 3,400 (Grade 1) | 7,500 |
| Granulocytes | 44% | 38% | 31% |
| (neutrophils) | | | |
| % of leukocytes | | | |
| Platelets (1,000/mm³) | 57 (Grade 2) | 106 (Grade 0) | 624 |
| SGOT transaminase | 172 (Grade 2) | 196 (Grade 2) | 84 |
| (U/L) | | | |
| SGPT transaminase | 24 | 37 | 42 |
| (U/L) | | | |
| Alkaline phosphatase | 126 | 197 | 105 |
| (U/L) | | | |
| Bilirubin (mg %) | 0.71 | 0.64 | 0.71 |
| Blood urea (mg %) | 34 | 24 | 38 |
| Blood uric acid (mg %) | 0.08 | 0.28 | 1.7 |
| Creatinine (mg %) | 0.61 | 0.46 | 0.33 |

### Pharmacokinetics in Rats

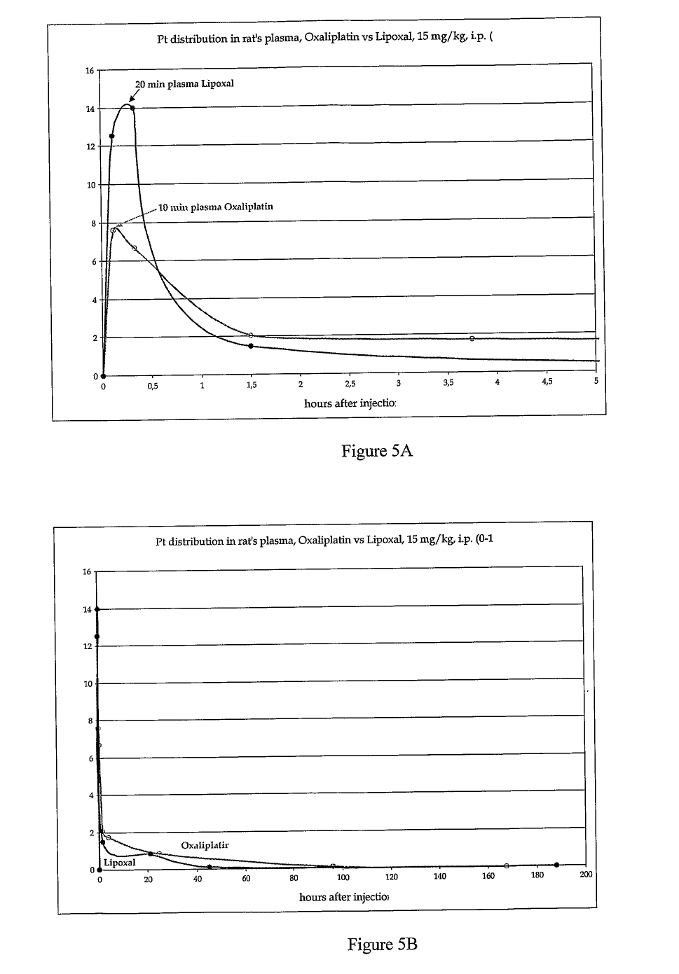

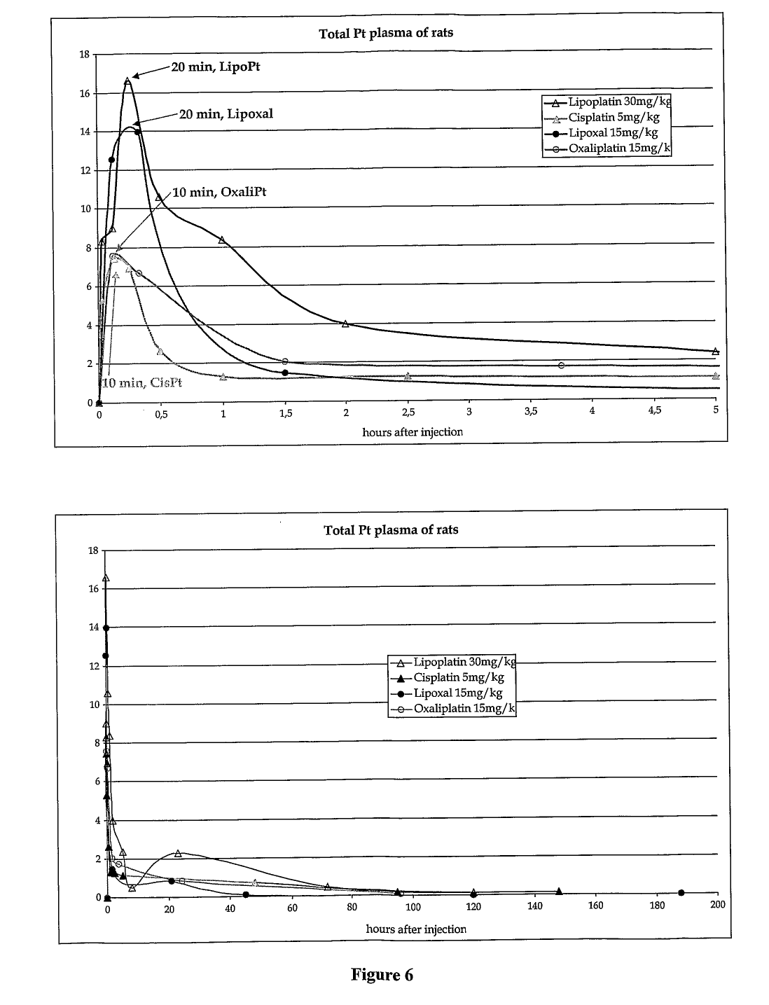

Rats were injected in the intraperitoneal cavity directly from a stock solution of 3 mg/ml liposomally encapsulated oxaliplatin or 3 mg/ml free oxaliplatin in 5% Dextrose to a final dose of 15 mg/Kg i.p. liposomally encapsulated oxaliplatin or oxaliplatin. At various time points postinjection blood was drawn, plasma was isolated and total platinum levels were measured for pharmacokinetic studies. FIG. 5A shows maxima levels of ˜14 mg total platinum/ml plasma after liposomally encapsulated oxaliplatin compared to ˜8 mg total platinum/ml plasma after free oxaliplatin and these were reached at 20 min for liposomally encapsulated oxaliplatin and at 10 min for free oxaliplatin. At approximately 45 min both groups exhibited similar levels of total platinum (˜5 mg total platinum/ml plasma) whereas at 4-5 h postinjection levels below 1 mg total platinum/ml plasma were obtained for liposomally encapsulated oxaliplatin compared to ˜2 mg total platinum/ml plasma for free oxaliplatin. At 40 h the levels of total platinum in rat plasma dropped to zero for liposomally encapsulated oxaliplatin and to ˜1 mg total platinum/ml plasma for free oxaliplatin; total platinum levels in rat plasma reached zero at ˜100 h post-injection for free oxaliplatin (FIG. 5B).

TABLE 3
Plasma

| | AUC | Cₘₐₓ | | Kel | t_{1/2} | Vss |
|---|---|---|---|---|---|---|
| Lipoxal ® | (h · μg/ml) | (μg/ml) | Cl (ml/g · h) | (1/h) | (h) | (ml/g) |
| (15 mg/Kg) | 53.7 s | 14.0 | 0.28 | 0.07 | 10.2 | 4.11 |

Kidney

| Lipoxal ® | AUC | | | Kel | t½ | |
|---|---|---|---|---|---|---|
| (15 mg/Kg) | (h * μg/g) | Cₘₐₓ (μg/g) | Cl (1/h) | (1/h) | (h) | Vss |
| | 460.1 | 13.7 | 0.03 | 0.033 | 21.00 | 1.00 |

Plasma

| Dose | AUC | Cmax | Cl | Kel | t½ | Vss |
|---|---|---|---|---|---|---|
| (mg/Kg) | (h * μg/ml) | (μg/ml) | (ml/g · h) | (1/h) | (h) | (ml/g) |
| 15 | 74.4 | 7.6 | 0.20 | N/A | N/A | N/A |

Kidney

| Dose | AUC | Cmax | | Kel | t½ | |
|---|---|---|---|---|---|---|
| (mg/Kg) | (h * μg/g) | (μg/g) | Cl (1/h) | (1/h) | (h) | Vss |
| 15 | ###### | 10.5 | 0.01 | 0.002 | 346.50 | 7.18 |

Mean pharmacokinetic parameters for total platinum calculated for the 15 mg/Kg dose of liposomally encapsulated oxaliplatin (Lipoxal®) or free oxaliplatin are shown in Table 2.

The AUC, determined using the linear trapezoidal method with extrapolation to infinity (Gibaldi et al, 1982 Gibaldi M, Perrier D: Noncompartmental analysis based on the statistical moment theory. In Pharmacokinetics, Gibaldi M, Perrier D (eds), pp 409-417, 2nd edn. Marcel Dekker: New York, 1982.), was 53.7 mg.h/ml for liposomally encapsulated oxaliplatin compared to 74.4 mg.h/ml for oxaliplatin.

The maximum concentration of total platinum in plasma reached (Cmax) was 14.0 mg/ml for liposomally encapsulated oxaliplatin compared to 7.6 mg/ml for free oxaliplatin. Total body clearance (Cl) was 0.28 ml/g.h for liposomally encapsulated oxaliplatin compared to 0.20 ml/g.h for free oxaliplatin. This was calculated from Cl=Di.v./AUC, where Di.v. is the i.p. dose of liposomally encapsulated oxaliplatin or free oxaliplatin and AUC the relative area under the curve for this specific dose.

The elimination rate constant (Kel) was 0.07 h-1 for liposomally encapsulated oxaliplatin. This was calculated by linear regression analysis of the logarithmic plasma concentration-time curve by the formula Kel=[Ln(Cp1)−Ln(Cp2)]/(t2−t1) where t1 and t2 are the starting and ending time points of measurements and Cp1 and Cp2 the starting and ending concentrations of total platinum in plasma for t1 and t2, respectively.

The elimination half-life (t½) was 10.2 h for liposomally encapsulated oxaliplatin. This was calculated by the formula: t½=0.693 (1/Kel). 1/Kel is the mean residence time (MRT), the statistical moment analogy to half-life t½ (Gibaldi et al, 1982).

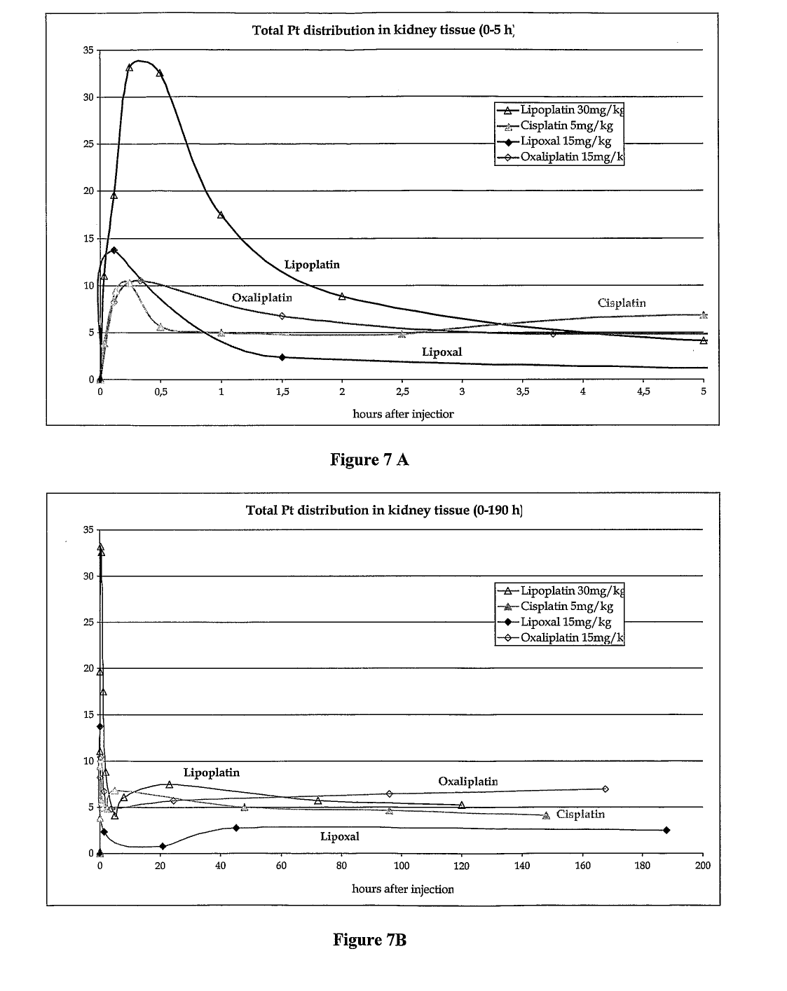

Total platinum levels in rat plasma were also determined in animals treated with Lipoplatin®, a different liposomal platinum drug currently under Phase III evaluation (Stathopoulos et al, 2005). Lipoplatin®, a liposomal cisplatin, was given at 30 mg/Kg i.p. The maxima levels were ˜17 mg total platinum/ml plasma after 30 mg/Kg Lipoplatin and these were reached at 20 min from injection in a similar time frame to liposomally encapsulated oxaliplatin (FIG. 6A). Cisplatin as a control was also administered i.p. to rats at its maximum tolerated dose of 5 mg/Kg; the maxima levels were ˜7.5 mg total platinum/ml plasma after cisplatin and these were reached at 10 min from injection in a similar time frame to oxaliplatin. All four drugs gave parallel pharmacokinetic behavior after ˜1.5 h post-injection; however, at 5 h Lipoplatin® injection resulted in ˜2.5 mg total platinum/ml plasma, followed by oxaliplatin at 2.0 mg total platinum/ml plasma, cisplatin at ˜1.5 mg total platinum/ml plasma and liposomally encapsulated oxaliplatin at ˜1.0 mg total platinum/ml plasma.

Biodistribution of Total Platinum in Rat Tissues after Liposomally Encapsulated Oxaliplatin or Free Oxaliplatin I.P. Infusion

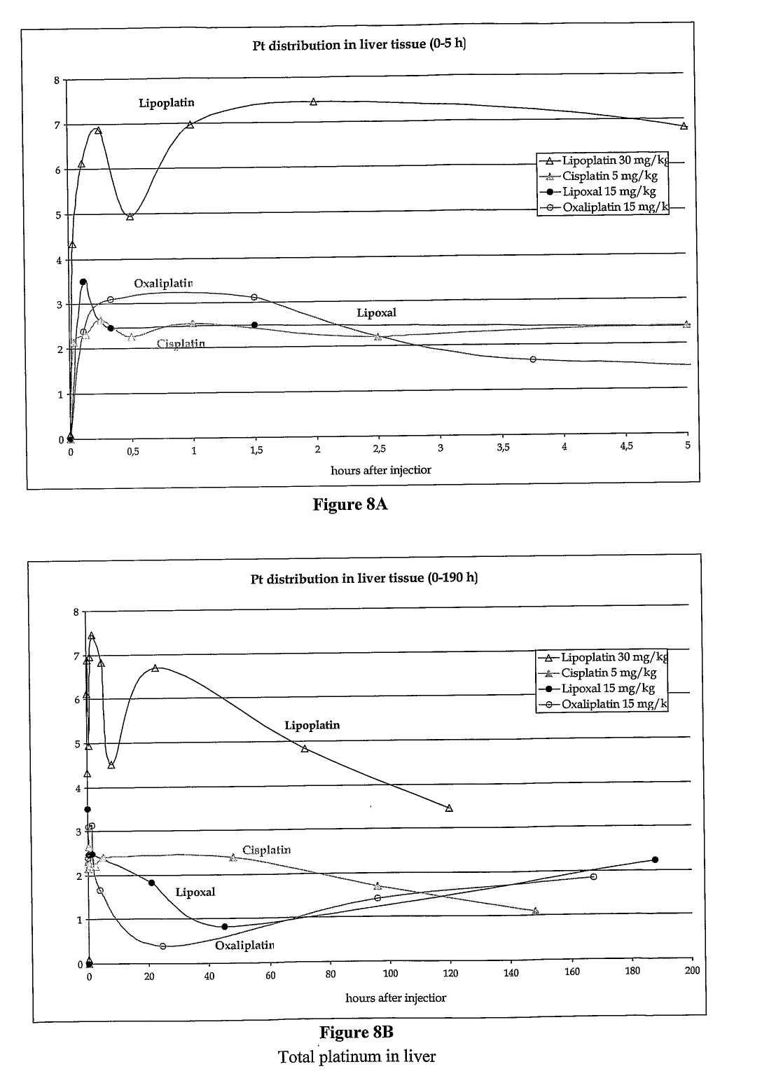

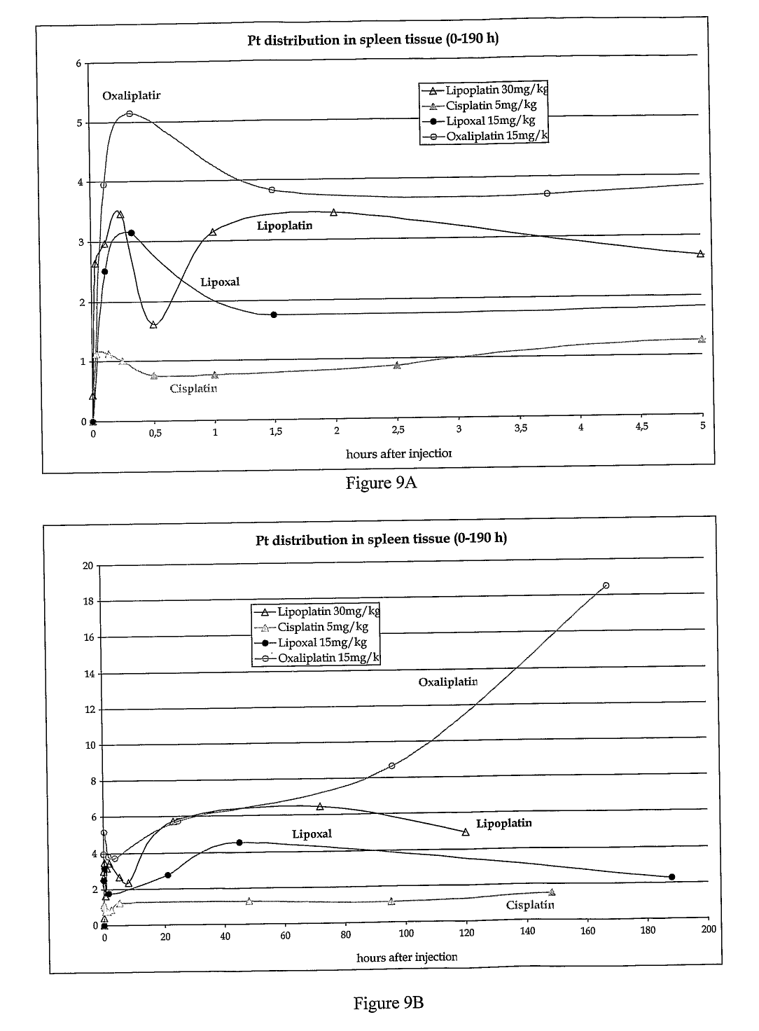

It is useful to study platinum drug distribution in mouse or rat tissue because of the accuracy of results and the relatively ease of assay of platinum with atomic absorption. Platinum levels in kidney: The maximum amount of total platinum in the kidney was 13.7 mg/g tissue after 15 mg/Kg liposomally encapsulated oxaliplatin compared to ˜10.5 mg/g tissue after 15 mg/kg oxaliplatin and was reached in 7-20 min from injection (FIG. 7A). However, after about 4 h the Pt levels in the kidney reached a minimum of 4.8 mg/g tissue after oxaliplatin and slightly increased to 6.9 mg/g tissue at 167 h postinjection. After liposomally encapsulated oxaliplatin treatment there is also a minimum of ˜1 mg/g tissue total Pt in the kidney reached at ˜20 h postinjection that slightly increased to 2.5 mg/g tissue at 188 h. Thus, kidneys display about 3 times higher levels of Pt after oxaliplatin compared to same dose of liposomally encapsulated oxaliplatin treatment at ˜7 days postinjection (FIG. 7B).

For comparison, Lipoplatin® at 30 mg/Kg reached maximum levels in kidney of 34 mg/g tissue compared to 10 mg/g tissue after 5 mg/Kg cisplatin.

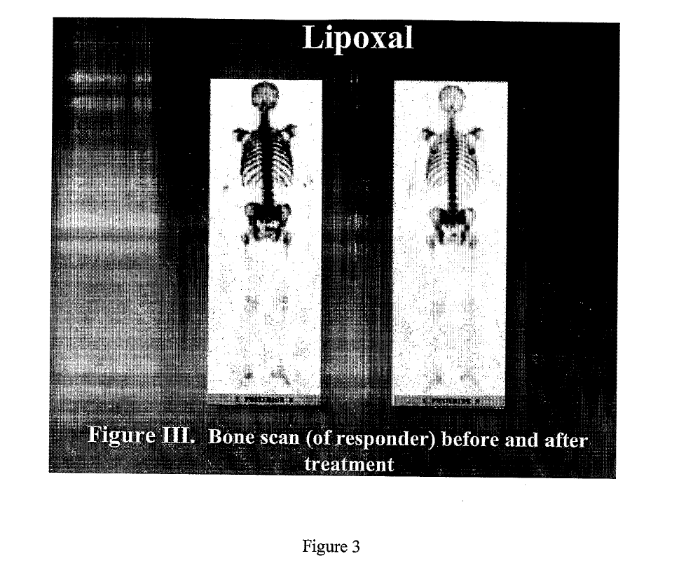

The pharmacokinetics in kidney exhibit a similar behavior between Lipoplatin® and liposomally encapsulated oxaliplatin. The maxima are 34 and 14 mg/g tissue for 30 mg/Kg Lipoplatin® and 15 mg/Kg liposomally encapsulated oxaliplatin respectively. This advocates for the similarity in kidney biodistribution of the two drugs that share common shell but differ in the drug they confine in their interior and in the tumors that they target. At 120 h the levels of total platinum in kidney are 5 mg/g tissue for 30 mg/Kg Lipoplatin® compared to ˜2.5 mg/g tissue for 15 mg/Kg liposomally encapsulated oxaliplatin (FIG. 3B). At ˜140 h postinjection the total platinum is ˜7 mg/g tissue after 15 mg/Kg free oxaliplatin compared to ˜4 mg/g tissue after 5 mg/Kg cisplatin (FIG. 3B).

### Conclusion:

Levels of Pt in kidneys were the highest among all rat tissues at 7 days followed by liver and spleen.

### Platinum Levels in Liver

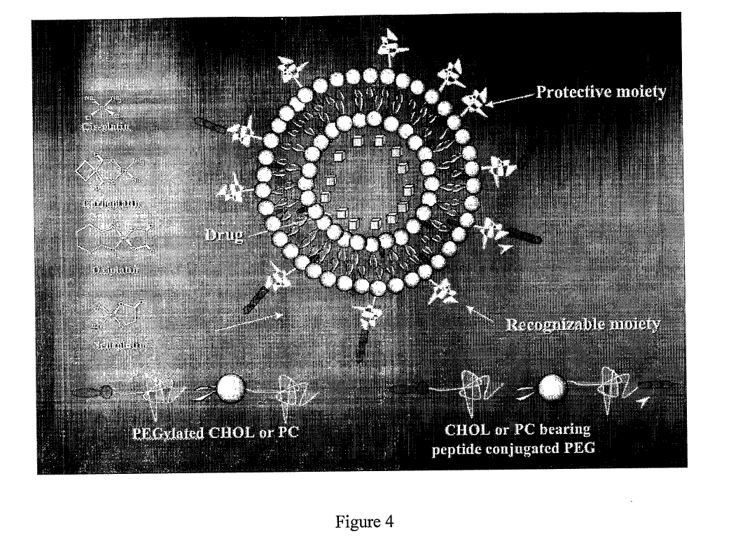

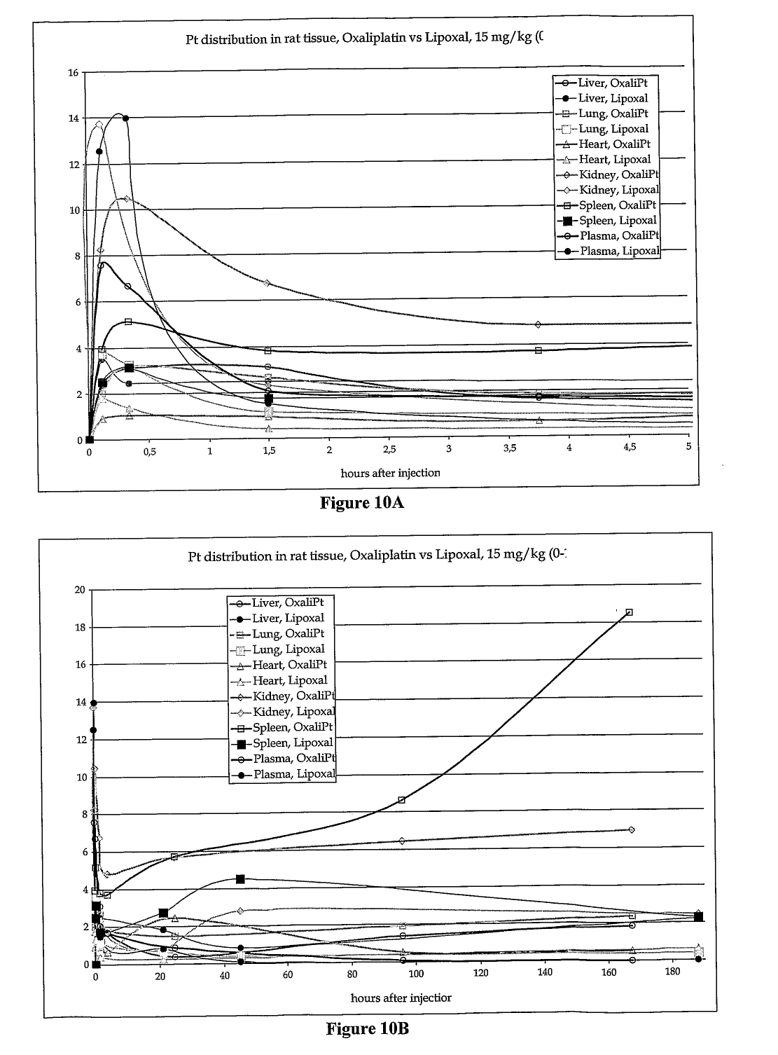

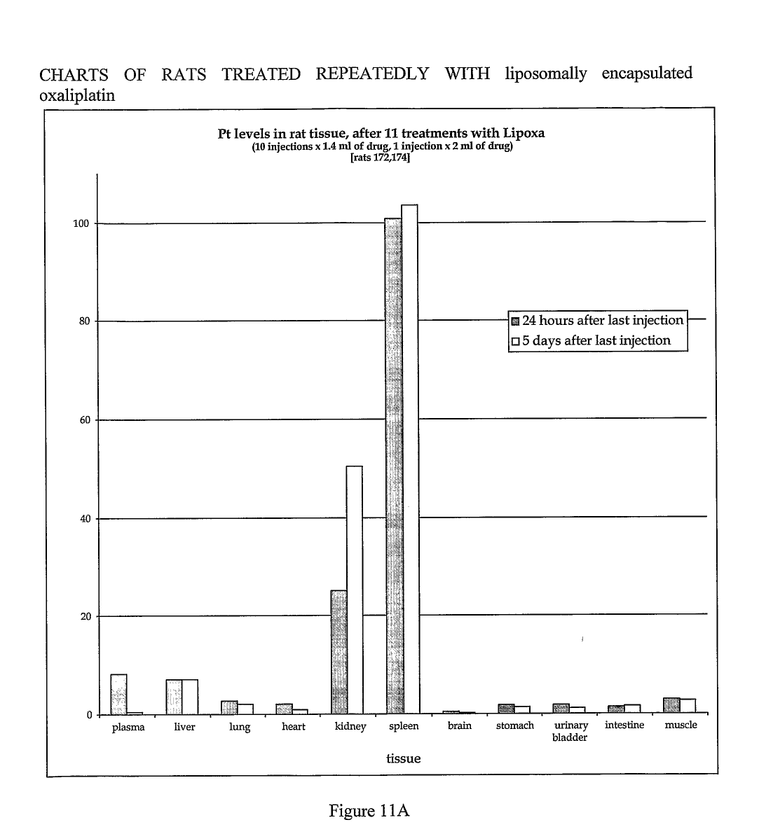

Total platinum in liver after 15 mg/Kg liposomally encapsulated oxaliplatin was 3.5 mg/g tissue attained at ˜7-10 min from i.p. infusion with an abrupt drop to 2.5 at 20 min and thereafter that was maintained for 5 h (FIG. 8A). On the contrary, infusion of the intraperitoneal cavity of rats with free oxaliplatin at the same dose resulted in similar total platinum levels in liver (3.0-3.5 mg/g tissue) that were attained at about 30 min from infusion, maintained for 2 h and then gradually decreased to 1.5 mg/g tissue at 5 h (FIG. 4A). Unlike plasma whose platinum levels drop to zero after about 40 h there was a liver accumulation of platinum of ˜2 mg/g tissue at 170-190 h after both liposomally encapsulated oxaliplatin and free oxaliplatin treatment (FIG. 8B).

For comparison, total platinum in liver after 30 mg/Kg Lipoplatin® was 7 mg/g tissue maintained for ˜5 h (FIG. 8A) it dropped to 4.5 at ˜12 h, increased to 6.5 at 24 h and gradually decreased to 3.5 at 120 h (FIG. 4B). Cisplatin also displayed a similar pattern to Lipoplatin® with a maximum level of 2.5 attained at 20 min maintained for 5 h (FIG. 4A), then dropped gradually to 1 from 24 to 150 h (FIG. 8B).

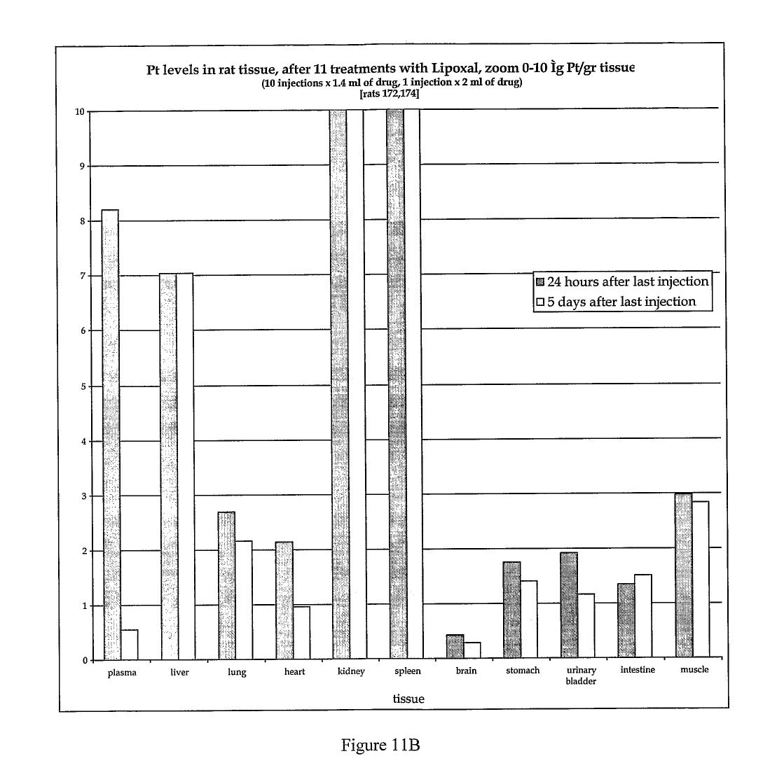

Total platinum in spleen: The maximum amount of total platinum in the spleen was 3.2 mg/g tissue following administration of liposomally encapsulated oxaliplatin at 15 mg/Kg compared to ˜5.2 mg/g tissue after 15 mg/Kg oxaliplatin and was reached in 15-20 min from injection (FIG. 9A). Up to ˜5 h postinjection there is a slight decrease ˜2 and ˜4 mg/g tissue following administration of liposomally encapsulated oxaliplatin vs oxaliplatin respectively. Thereafter there is an increase in the spleen levels of total platinum after liposomally encapsulated oxaliplatin up to ˜45 h to ˜4.5 mg/g tissue followed by a decline to ˜2 mg/g tissue at 190 h. On the contrary, there is a continuous accumulation of total platinum in the spleen following free oxaliplatin infusion that reaches 18.5 mg/g tissue at 168 h (FIG. 9B). This is accompanied by tremendous loss in spleen weight at 7 days presumably as a result of apoptotic death of spleenocytes from the toxicity to free oxaliplatin. In fact, for a mouse of an average body weight of 150 g before study the final body weight at 7 days was 109 g and the weight of the spleen was 0.188 g.

There was congestion (accumulation of blood) in the spleen of animals treated with liposomally encapsulated oxaliplatin.

However, after about 1 h the Pt levels in the kidney were higher from free oxaliplatin than liposomally encapsulated oxaliplatin treatment; they reached a minimum at 12-24 h (5 mg/g tissue after oxaliplatin, 1 mg/g tissue after liposomally encapsulated oxaliplatin) and started increasing again; at 170 h postinjection kidney tissue displayed 7 mg Pt/g tissue after oxaliplatin and 2.5 mg Pt/g tissue after liposomally encapsulated oxaliplatin (FIG. 5B).

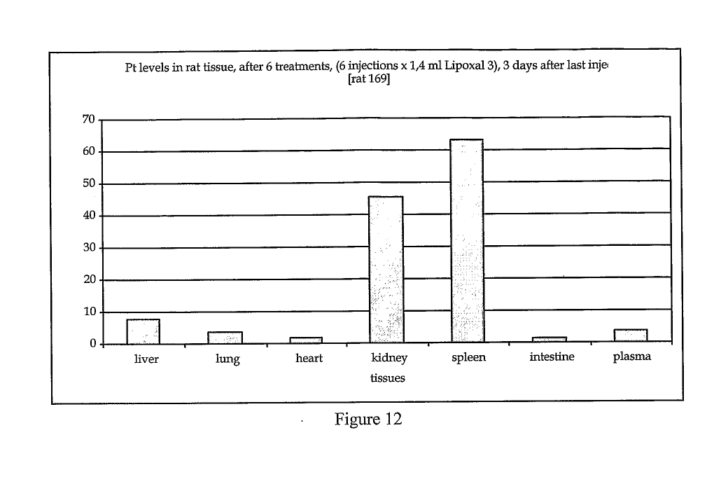

The comparative measurements of total platinum in all rat tissues examined after liposomally encapsulated oxaliplatin or free oxaliplatin are summarized in FIG. 10. Plasma levels after 15 mg/Kg oxaliplatin at 20 min from injection attained the highest level of total platinum (14.2 mg/ml) among all tissues; kidney tissue had a comparable high level at ˜10 min following i.p. injection of liposomally encapsulated oxaliplatin (13.8 mg/ml) (FIG. 10A). The next levels include kidney platinum after oxaliplatin and plasma after oxaliplatin. Spleen appears to be the next higher level (5 mg/g tissue after 15 mg/Kg oxaliplatin) a level that continuously increases and is becomes the highest after 24 h and even higher at 170 h (18.5 mg/g tissue). Therefore, overall, spleen finally accumulates the highest level of platinum after oxaliplatin. In this respect the difference between platinum accumulation in spleen after free oxaliplatin or liposomally encapsulated oxaliplatin is obvious (FIG. 10B).

Maximum levels of platinum (in mg Pt/ml plasma or per g tissue) in rat tissues (attained at 7-20 min) following i.p. injection of 4 drugs. (ND, not determined).

TABLE 4
Comparison of total platinum levels in various rat tissues after Liposomal
Oxaliplatin, free Oxaliplatin, Liposomal Cisplatin, and free Cisplatin at
7-20 min, 5 h and 5-7 days postinjection

| | Liposomally | | | |
|---|---|---|---|---|
| | Encapsulated | | | |
| | Oxaliplatin | Oxaliplatin | Lipoplatin ® | Cisplatin |
| Drug & dose | 15 mg/Kg | 15 mg/Kg | 30 mg/Kg | 5 mg/Kg |
| Kidney | 13.7 | 10.5 | 33.2 | 10.2 |
| Plasma | 14.0 | 7.6 | 16.6 | 7.5 |
| Liver | 3.5 | 3.1 | 6.9 | 2.7 |
| Spleen | 3.1 | 5.1 | 3.5 | 1.1 |
| Lung | 3.7 | 3.2 | 7.5 | 2.0 |
| Heart | 1.8 | 1.1 | 4.5 | 0.8 |
| Brain | ND | ND | 1.7 | 0.3 |

5 h

| | Liposomally | | | |
|---|---|---|---|---|
| | Encapsulated | | | |
| | Oxaliplatin | Oxaliplatin | | |
| | 15 mg/Kg | 15 mg/Kg | Lipoplatin | Cisplatin |
| Drug & dose | estimation | estimation | 30 mg/Kg | 5 mg/Kg |
| Kidney | 2.0 | 5.0 | 4.2 | 6.8 |
| Plasma | 0.9 | 1.7 | 2.4 | 1.1 |
| Liver | 2.4 | 1.5 | 6.8 | 2.4 |
| Spleen | 1.8 | 3.8 | 2.7 | 1.2 |
| Lung | 0.8 | 1.7 | 0.6 | 0.7 |
| Heart | 0.3 | 0.8 | 0.2 | 0.2 |
| Brain | ND | ND | 0.1 | 0.1 |

5-7 days

| | Liposomally | | | |
|---|---|---|---|---|
| | Encapsulated | | | |
| | Oxaliplatin | Oxaliplatin | Lipoplatin | Cisplatin |
| Drug & dose | 15 mg/Kg | 15 mg/Kg | 30 mg/Kg | 5 mg/Kg |
| Kidney | 2.5 | 6.9 | 5.2 | 4.1 |
| Plasma | 0 | 0 | 0.1 | 0.1 |
| Liver | 2.2 | 1.8 | 3.5 | 1.1 |
| Spleen | 2.3 | 18.5 | 4.9 | 1.5 |
| Lung | 0.4 | 2.4 | 0.2 | 0.3 |
| Heart | 0.6 | 0.5 | 0.1 | 0.1 |
| Brain | ND | ND | 0.1 | 0 |

Kidney, Spleen and Liver have significant Pt levels at 5-7 days post-treatment with liposomally encapsulated oxaliplatin.

Spleen Kidney Lung and Liver have significant Pt levels at 5-7 days post-treatment with free Oxaliplatin.

Kidney, Spleen and Liver have significant Pt levels at 5-7 days post-treatment with Lipoplatin®.

Kidney, Spleen and Liver have significant Pt levels at 5-7 days post-treatment with Cisplatin.

The data show that after 15 mg/Kg i.p. liposomally encapsulated oxaliplatin compared with 15 mg/Kg i.p. free Oxaliplatin:

1. the plasma levels in total platinum are 14 mg/ml plasma after liposomally encapsulated oxaliplatin,
the plasma levels in total platinum are 7.6 mg/ml plasma after Oxaliplatin
Maxima are reached in about 7-20 min from i.p. injection
This shows longer circulation of liposomally encapsulated oxaliplatin compared to Oxaliplatin
2. Levels in kidney are higher with liposomally encapsulated oxaliplatin (14 mg/g tissue) compared to free Oxaliplatin (11 mg/g) in the initial 15 min from injection but at 1.5 h and thereafter levels in kidney become higher with free Oxaliplatin (6.7 mg/g tissue) compared to liposomally encapsulated oxaliplatin (2.3 mg/g) at 1.5 h.
3. Levels in spleen are higher with free Oxaliplatin (3.8 mg/g tissue) compared to liposomally encapsulated oxaliplatin (1.8 mg/g) at 1.5 h postinjection.
4. Levels in heart are comparable and low between the two drugs

Platinum levels in plasma: The maximum amount of total platinum in the plasma is 14 mg/ml after 15 mg/Kg i.p. liposomally encapsulated oxaliplatin compared to ˜7.5 mg/ml tissue after 15 mg/Kg Oxaliplatin and is reached in 7-20 min from injection (FIG. 10A). However, after about 1 h the Pt levels in the plasma become higher from free oxaliplatin than from liposomally encapsulated oxaliplatin treatment, and this is maintained throughout the rest of the curve up to 50 h where the levels for liposomally encapsulated oxaliplatin become zero and up to ˜100 h when the levels for free Oxaliplatin become zero.

Platinum levels in kidney: The maximum amount of total platinum in the kidney is 13.5 mg/g tissue after 15 mg/Kg Lipoxal compared to ˜10.5 mg/g tissue after 15 mg/Kg Oxaliplatin and is reached in 15-20 min from injection (FIG. 10A). However, after about 4 h the Pt levels in the kidney reach a minimum of 4.8 mg/g tissue after free oxaliplatin and slightly increase to 6.9 g/g tissue at 167 h postinjection. After liposomally encapsulated oxaliplatin treatment there is also a minimum of ˜1 mg/g tissue total Pt in the kidney reached at ˜20 h postinjection that slightly increases to 2.5 mg/g tissue at 188 h. Thus, kidneys display about 3 times higher levels of Pt after free oxaliplatin compared to same dose of liposomally encapsulated oxaliplatin treatment at ˜7 days postinjection. Levels of Pt in kidneys are the highest among all rat tissues at 7 days followed by liver and spleen.

Platinum levels in spleen: The maximum amount of total platinum in the spleen is 14 mg/g tissue after 15 mg/Kg liposomally encapsulated oxaliplatin compared to ˜7 mg/g tissue after 15 mg/Kg free Oxaliplatin and is reached in 15-20 min from injection (FIG. 10A). However, after about 1 h the Pt levels in the kidney are higher from free oxaliplatin than liposomally encapsulated oxaliplatin treatment they show a minimum around 12-24 h (5 mg/g tissue after oxaliplatin, 1 mg/g tissue after liposomally encapsulated oxaliplatin) and start increasing again; at 170 h postinjection kidney tissue displays 7 mg Pt/g tissue after free oxaliplatin and 2.5 mg Pt/g tissue after liposomally encapsulated oxaliplatin (FIG. 10A).

### Drug Treatment Resulting Weight Differences and Reduction in Tissue Size

Animals which were treated with liposomally encapsulated oxaliplatin (15 mg/kg) and free Oxaliplatin (15 mg/kg) and were sacrificed 7.8 and 7 days after i.p. injection of the drug, exhibit some great differences in both total weigh loss and weight of individual organs.

TABLE 5
Reduction in body weight as a result of cahexia after oxaliplatin
treatment. The animals treated with comparable doses of liposomal
oxaliplatin exhibit less overall weight or organ weight reduction.
Spleen appears to be the tissue affected the most by free oxaliplatin.

| | Liposomal Oxaliplatin | Oxaliplatin |
|---|---|---|
| Drug | 15 mg/kg | 15 mg/kg |
| Time elapsed after i.p. | 7.8 days | 7 days |
| administration | | |
| Animal weight | 167 gr | 106 gr |

| | |
|---|---|
| Organ | Weight of total animal organ (g) |

| | | |
|---|---|---|
| Lung | 1.059 | 0.934 |
| Heart | 0.604 | 0.523 |
| Kidney | 0.645 | 0.475 |
| Spleen | 0.617 | 0.188 |

Animal treated with free Oxaliplatin, exhibit a great weight loss during the 7 days after drug administration, which is estimated to be over 40 gr of total body weight at time of treatment. Furthermore, there is a significant reduction in the size of spleen tissue, which is mirrored to an extremely high value of Pt concentration (18.5 mg Pt/g of tissue).

Loss of appetite after free Oxaliplatin administration and drug toxicity, resulted weight loss and reduction in the size of spleen; those phenomena observed at animals sacrificed 7 days after drug administration and therefore exhibited high Pt concentration values at tissue charts of free Oxaliplatin at 7 days following I.P. injection.

Same impact could be considered to be regarding other tissue, as long as Pt concentration values at all free Oxaliplatin tissue charts (Liver, Lung, Heart, Spleen, Kidney) at time point: 7 days, are exhibiting an increase.

Mice injected with 5 mg/Kg oxaliplatin died of toxicity and the dose was lowered to 4 mg/Kg. The dose of Lipoxal was 16 mg/Kg i.v. and the toxicity was lower than 4 mg/Kg oxaliplatin. The anticancer efficacy of 4 mg/Kg oxaliplatin was lower than that of 16 mg/Kg Lipoxal in animals with human tumors.

### Example 2B

### A Phase I Clinical Study

The aim of the study was a) to estimate the adverse reactions and detect the dose limiting toxicity (DLT) as well as the maximum tolerated dose (MTD) of liposomally encapsulated oxaliplatin. Patients and methods: In total, 27 patients with advanced disease were included in the study. All patients were pretreated with the standard chemotherapy according to the established guidelines. At entry to the present trial all were on recurrent or progressive disease. All patients had gastrointestinal cancers of stage IV (colorectal, gastric and pancreatic cancers). We set six different dose levels of liposomally encapsulated oxaliplatin and in each level at least 3 patients were included. The dose levels were: 1) 100 mg/m² 2) 150 mg/m² 3) 200 mg/m2 4) 250 mg/m² 5) 300 mg/m² 6) 350 mg/m². Eight additional patients were treated at 300 mg/m² as an MTD. Treatment was given once weekly for three consecutive weeks repeated every 4 weeks. Results: No serious side effects were observed in the first four dose levels (100-250 mg/m²). At levels 5 and 6 mild myelotoxicity and nausea were seen. The most common adverse reaction was peripheral neuropathy of grade II and was observed in all 4 patients treated at 350 mg/m². We, therefore, considered DLT the 350 mg/m2 level and MTD the 300 mg/m² level. Of the 27 patients, three showed partial response and 18 patients had stable disease for 4 months, median range (2-9). Conclusion: In the present Phase I study we found that the most common toxicity is peripheral neuropathy at the 300 and 350 mg/m² dose levels. Liposomally encapsulated oxaliplatin is well tolerated and reduces significantly all other side effects of free oxaliplatin especially myelotoxicity and G.I. tract toxicities. These preliminary results showed adequate effectiveness in pretreated patients.

The said study was a clinical trial with liposomally encapsulated oxaliplatin (Lipoxal®) with the following primary objectives: a) to define the dose limiting toxicity (DLT) and maximum tolerated dose (MTD) of escalating doses of a weekly Lipoxal administration, b) to detect the toxicity profile and pharmacokinetics of lipoxal monotherapy in pretreated advanced G.I. tract cancer patients. Secondary objectives were the efficacy and survival.

### Patients and Methods

The study was a phase I cohort, dose-escalation trial of liposomally encapsulated oxaliplatin. The study protocol was reviewed and approved by our Institutional Review Board. An informed consent document satisfying all institutional requirements was read by the patients and signed as a condition of their registration.

### Eligibility Criteria

All patients were required to meet the following criteria: confirmed histologic or cytologic diagnosis of cancer, at least one bidimensionally measurable or evaluable disease, WHO performance status 0-2, a life expectancy greater than 3 months, previous treatment by standard or first-line chemotherapy and at the time of entry to have been refractory to any prior cytotoxic treatment. Patients were eligible if they had had two or three previous courses, provided that they had been off treatment for at least 3 weeks.

### Assessment

Eligible patients over 18 years of age were required to have adequate hematologic, renal and hepatic functions as defined by WBC count 3.5×109/l, absolute neutrophil count 1.5×109/l, platelet count 100×109/l, hemoglobin level 9 g/dl, total bilirubin level 1.5 mg/dl, ALT and AST twice the upper normal limit in the absence of liver metastases or five times the upper normal limit in case of documented liver metastasis and creatinine level 1.5 mg/dl. Medical history, physical examination, assessment of vital signs, electrocardiogram, chest, and abdominal computed tomography (or ultrasound) were performed before treatment. During treatment (1 day before each course) blood count, blood urea and sugar, serum creatinine and uric acid tests, and ECG were done. CT scan assessments were done after at least eight weekly drug infusions, or earlier—on disease progression.

### Treatment

Drug characteristics: Provided in 3 mg/ml, 50 ml per glass vial, 150 mg of oxaliplatin per glass vial. Store Liposomally encapsulated oxaliplatin at 4 degrees Celsius, opaque appearance. Characteristic of a Liposomal drug: Liposomally encapsulated oxaliplatin is diluted in 1 lt 5% dextrose and given at 3 hours intravenous infusion once weekly for 8 consecutive weeks. In case of side effects and in particular myelotoxicity or neurotoxicity delay of treatment administration would take place by one week. No pre- or post hydration was needed. No other drugs such as antihemetic or anti-allergic were planned to be given prophylactically. In case of nausea or vomit, support by antiemetics (Ondasetron) or antiallergic (Dexamethasone) were to be given.

In animal studies that preceded 400 mg/m2 to 600 mg/m2 approximately was defined as the MTD. In humans we decided to start at a dose of 100 mg/m2 for level one. The dosage increase was decided to be 50 mg/m2 per level. In table 1 the dose escalation of liposomally encapsulated oxaliplatin per group of patients is presented.

Drug-related toxicities were evaluated during each cycle of therapy and graded according WHO criteria. A DLT was defined as any Grade 3 or 4 toxicity, with neutrophil count <500 mm2 associated with fever persisting longer than 72 hours, in 50% of the patients. Other toxicity of Grade III and in particular neurotoxicity was also considered DLT if it was observed in at least 50% of the patients. One dose level less than that of DLT was defined as MTD. Cohorts of three patients at minimum were scheduled for entry at each dose level. Escalation of the dose to the next higher level proceeded after all three patients had received the first cycle of therapy with the preceding dose and each one was observed for at least 3 weeks without evidence of a DLT. Additional two patients were enrolled at a given dose level if the first patient of that level experienced a DLT, on the first period of 3 weeks, treatment. Treatment was discontinued with the occurrence of a DLT and the patient continued on one level below.

### Pharmacokinetics

For the pharmacokinetic study patients were bled at the following hours. 0 (before drug infusion and after infusion start 2, 4, 8, 24, 48, 72, 120 (5 days) and 168 (7 days) hours. 3 ml blood was drawn into EDTA or heparin-containing tubes, then was centrifuged and refrigerated at 2° C. and eventually were sent to the laboratory to be analyzed for total platinum levels. A sample of 5 patients was used. Platinum levels (total and serum ultrafiltrates) were measured with atomic absorption (Perkin Elmer AA 700 Graphite Furnace Atomic Absorption Spectrometer at Regulon A. E.). It was at certain dose levels . . . (200 mg and 300 mg/m2): the area under the plasma concentration-time curve (AUC), the Cmax (maximum concentration of total platinum in serum). The total body clearance (Cl) was calculated from CL=Div/AUC, where Div is the intravenous dose of Lipoplatin® and AUC the relative Area under curve for a specific dose. The Kel (elimination rate constant) was calculated by linear regression analysis of the logarithmic plasma concentration-time curve from the formula Kel=[Ln(Cp1)-Ln(Cp2)]/(t2-t1) where t1 and t2 are the starting and ending time points of measurements and Cp1 and Cp2 the starting and ending concentrations of total platinum in serum for t1 and t2 respectively.

The t½ (elimination half-time) was calculated from the formula t½=0.693 (1/Kel). 1/Kel is the MRT (Mean Residence Time), the statistical moment analogy to half-life t½ (Gibaldi et al. 1982). In effect, the MRT represents the time for 63.2% of the administered dose to be eliminated.

### Results

### Patients

The patient characteristics are shown in table 5. 27 patients were in total enrolled. Age 32-78, median age 62, male 18, female 9. P.S. 0-2. All the patients had been previously treated by chemotherapy. Previous treatments per tumor.

### Toxicity

Liposomally encapsulated oxaliplatin G.I. tract toxicity was negligible. Without antiemetics (Ondosetron), nausea or mild vomit was seen. But with ondasetron no nausea vomit was observed. No diarrhea also. Mild, of grade I myelotoxicity (neutropenia) was only seen in 2 patients (%) with the highest doses given (350 mg/m2). No hepatotoxicity, no renal toxicity, no cardiotoxicity, no alopecia was seen. Mild asthenia in 3 patients was seen.

The main side effect was neurotoxicity, which was seen after at least 3 infusions of the agents and was of grade I at the 3rd and 4th level and of grade 2 at the 5th level and grade 2 in 100% of the patients at level 6th.

On the basis of these results neurotoxicity of grade III was considered as the dose limited toxicity observed in 100% of patients treated with 350 mg/m2 of Liposomally encapsulated oxaliplatin. The one dose under 300 mg/m2 was defined as the maximum tolerated dosis (MTD). In table 5 the liposomally encapsulated oxaliplatin dose escalation and the number of patients treated at each of the six levels is presented.

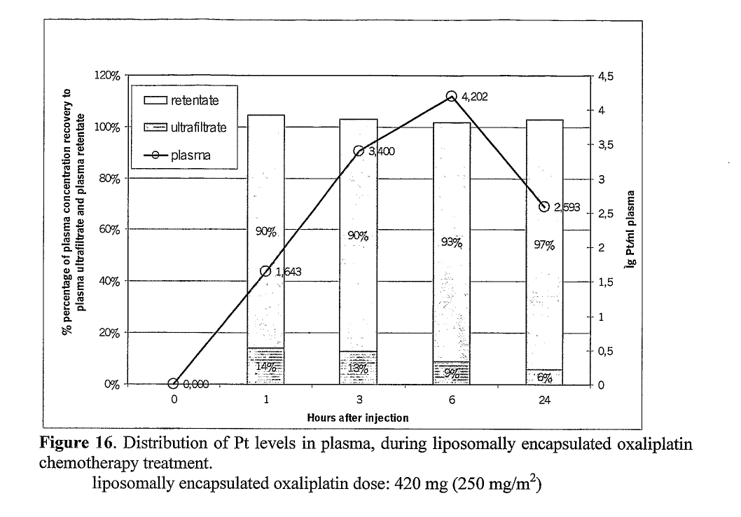

Pharmacokinetics: The results are represented in table 7 and in FIGS. 15 and 16. It was found that half life of oxaliplatin in plasma concentration was 24 hours and the urine excretion is integrated in 7 days.

Compliance with Treatment

A total number of 104 infusions (cycles) were administered with a median of 4 cycles per patient (ranging from 2-15). The median interval between cycles was 7 days. Dose intensity was 100% of the planned. No patient happened to have a treatment delay as no hematologic toxicity of grade III or IV was detected. Only patients with dosage 350 mg/m2 after the most 4 or 5 infusions (cycles) had a two weeks interval before they were classified to the lower dose of 300 mg/m2. Some patients stopped treatment due to disease progression after 4-6 cycles. This was applied in 17 patients (62.9%). Twelve patients were still alive at the end of the study (44.4%). The causes of death were disease progression.

### Responses to Treatment

Responses were analyzed on an intention-to-treat basis. There were no complete responses. 3 patients out of 27 (11.1%) showed partial response. These patients were 2 with gastric cancer, one with pleural effusion and the other with bone metastases; the third was a patient with liver metastases from colon carcinoma. The detection of partial response was based on CT-scan for the 1st patient, with bone scan on the second patient and for the third patient with CT-scan and bilirubin serum level. Two figures are presented: FIG. 1 bone scan before and after treatment for the 2nd patient and bilirubin serum level curve in the 3rd patient. Exceptionally, we treated the third patient while serum bilirubin level was 51 mg/dl, which after 2 courses the level dropped to 8 mg/dl and lasted for 5 weeks.

The duration of response was 4, 7, 2 months for each patient respectively. 18 patients showed stable disease (66.66%) with a median duration of 4 months (range 2-9 months). 5 of the patients could be classified, according to a non valid anymore, classification, to minor responses. 6 patients showed disease progression. In all the 3 responders there was also a reduction by 50% or more of the marker CA-19-9. Also, the performance status level was improved from 2 to 1 in all the 3 responders.

### Conclusion

Liposomally encapsulated oxaliplatin has been tested in the present trial (example) as a monotherapy (single treatment) in patients with advanced cancer of the gastrointestinal system. All patients were pretreated by a standard treatment and all the included colorectal patients had also been treated by free oxaliplatin. This treatment with liposomally encapsulated oxaliplatin had only been tested before in preclinical studies. No other clinical trial was previously performed. The present trial was based on the data of the preclinical studies and on the experience and data of the non-liposomal (free) oxaliplatin. The last was mainly helping in focusing our present trial in estimating the similarities or differences of the liposomally encapsulated oxaliplatin versus the bare (free) oxaliplatin. G.I. tract and hematologic side effects were shown to be greatly reduced. The only side effect that remained without any difference—any reduction, was the neurotoxicity. That was seen often, increased more or less analogously with the increase of the agent dosage and acted as the only or main criterion for defining the dose limiting toxicity. The MTD defined dosis was 300 mg/m2 administered weekly. There was also an additive neurotoxicity as also it happens with non-liposomal oxaliplatin (ref.). In respect of effectiveness the 11% of response rate observed in pretreated patients refractory to previous established tumors could have some meaning for future trials in a combined chemotherapy modality. It is also important to point out that the cancer types selected for this trial are not of the most sensitive ones to chemotherapy.

This study has established an MTD and further investigation is needed in particular with other agents in combination.

As a result, this example shows that liposomal oxaliplatin is a well tolerated agent. The dosis 300 mg/m2 was defined as MTD. The GI-tract and bone marrow toxicities are very much reduced compared to the bare form of oxaliplatin. The only adverse reaction that remains is the neurotoxicity which is the one that defines the DLT.

TABLE 6
Liposomally encapsulated oxaliplatin (Lipoxal ®) dose escalation

| Dose level | Number of patients | Lipoxal ® (mg/m² per week) |
|---|---|---|
| I | 3 | 100 |
| II | 3 | 150 |
| III | 5 | 200 |
| IV | 4 | 250 |
| V | 4 + 4 | 300 |
| VI | 4 | 350 |

Lipoxal
Lipoxal dose escalation

| Dose | Number of | Lipoxal |
|---|---|---|
| level | patients | (mg/m² per week) |
| I | 3 | 100 |
| II | 3 | 150 |
| III | 5 | 200 |
| IV | 4 | 250 |
| V | 4 + 4 | 300 |
| VI | 4 | 350 |

TABLE 7
Baseline patients' characteristics

| | N | (%) |
|---|---|---|
| Patients total | 27 | (100) |
| Age, years | | |

| | |
|---|---|
| Median | 62 |
| Range | 32-78 |
| Gender | |

| | | |
|---|---|---|
| Male | 18 | (66.66) |
| Female | 9 | (33.33) |
| PS | | |
| 0 | 2 | (7.40) |
| 1 | 14 | (51.85) |
| 2 | 11 | (40.74) |
| Stage | | |
| IV | 27 | (100) |
| Primary tumor | | |
| Colorectal | 12 | (44.44) |
| Pancreas | 8 | (29.62) |
| Stomach | 4 | (14.81) |
| Biliary | 2 | (7.41) |
| Liver | 1 | (3.70) |
| Histology | | |
| Adenocarcinoma | 27 | (100) |

| | |
|---|---|
| Stage of disease | |
| IV | 27 (100) |
| Primary tumor | |
| Colorectal | 12 (44.44) |
| Pancreas | 8 (29.62) |
| Stomach | 4 (14.81) |
| Bilary | 2 (7.41) |
| Liver | 1 (3.70) |
| Histology | |
| Adenocarcinoma | 27 (100) |
| Previous chemotherapy | |

TABLE 8
Plasma pharmacokinetic parameter estimates for Lipoxal in patients
(see text for definitions of parameters)

| Dose | Cₘₐₓ | AUC | Cl | Kₑₗ | t_{1/2} | Vₛₛ |
|---|---|---|---|---|---|---|
| (mg/m²) | (μg Pt/ml) | (μg Pt * h/ml) | (L/h * m²) | (1/h) | (h) | (L/m²) |
| 250 | 9.175 | 424.4 | 0.289 | 0.028 | 24.3 | 9.7 |
| 350 | 12.087 | 782.3 | 0.219 | 0.020 | 35.5 | 10.9 |

| | | Number of | | |
|---|---|---|---|---|
| | | patients | | |
| | Dose | grade | | |
| Toxicity | level | I | II | III |
| Nausea vomit | II | 2 | — | — |
| (without . . . ) | | | | |
| Neurotoxicity | III | 2 | — | — |
| Neurotoxicity | IV | 3 | — | — |
| Neurotoxicity | V | 2 | 3 | — |
| Neurotoxicity | VI | 1 | 2 | 1 |
| Renal | All levels | — | — | — |
| Cardiotoxicity | All levels | — | — | — |
| Hepatotoxicity | All levels | — | — | — |
| Alopecia | All levels | — | — | — |
| Asthenia | All levels | — | — | — |

Lipoxal
Response to treatment

No CR was observed.
3 out 27 patients showed PR (11.1%)
Two of 3 were patients with advanced gastric cancer
( . . . ).
One was patient with colorectal cancer - liver
metastases - (jaundice).
Response duration 4, 7, 2 months.
18 patients stable disease (66.66%)
median duration 4 months (range 2-9)
5 patients progression disease

### REFERENCES

1. De Gramont A, Vignond J, Tournigand C, et al: Oxaliplatin with high-dose leucovorin and 5-flouorouracil 48-h continuous infusion in pretreated metastatic colorectal cancer. Eur J Cancer 33: 214-219, 1997.
2. Giaccheti S, Perpoint B, Zidani R, et al: Phase III multicentre randomized trial of oxaliplatin added to chronomodulated fluorouracil-leucovorin as first line treatment of metastatic colorectal cancer. J Clin Oncol 18: 136-147, 2000.
3. De Gramont A, Figer A, Seymour M, et al: Leucovorin and fluorouracil with or without oxaliplatin as first-line treatment in advanced colorectal cancer. J Clin Oncol 18: 2938-2947, 2000.
4. Giacchetti S, Perpoint B, Zidani R, et al: Phase III multicenter randomized trial of oxaliplatin added to chronomodulated fluorouracil-leucovorin as first-line treatment of metastatic colorectal cancer. J Clin Oncol 18: 136-147, 2000.
5. Souglakos J, Mavroudis D, Kakolyris S, et al: Triplet combination with irinotecan plus oxaliplatin plus continuous-infusion fluorouracil and leucovorin as first-line treatment in metastatic colorectal cancer: a multicenter phase II trial. J Clin Oncol 20: 2651-2657, 2002.
6. Scheithauer W, Kornek G V, Raderer M, et al: Randomized multicenter phase II trial of two different schedules of capecitabine plus oxaliplatin as first-line treatment in advanced colorectal cancer. J Clin Oncol 21: 1307-1312, 2003.
7. Goldberg R M, Sargent D J, Morton R F, et al: A randomized Controlled Trial of Fluorouracil plus Leucovorin, Irinotecan, and Oxaliplatin combinations in Patients with Previously Untreated Metastatic Colorectal Cancer. J Clin Oncol 22: 23-30, 2004.
8. Sorbye H, Glimelius B, Berglund A, et al: Multicenter Phase II study of Nordic Fluorouracil and Folinic Acid Bolus Schedule Combined with Oxaliplatin as First-Line Treatment of Metastatic Colorectal Cancer. J Clin Oncol 22: 31-38, 2004.
9. Cassidy J, Misset J L: Oxaliplatin-related side effects: characteristics and management. Semin Oncol 29 (Suppl 15), 11-20, 2002.
10. Cassidy J, Misset J L: Oxaliplatin-related side effects: characteristics and management. Semin Oncol 29 (Suppl 15), 11-20, 2002.
11. Jerremalm E, Eksborg S, Ehrsson H: Hydrolysis of oxaliplatin-evaluation of the acid dissociation constant for the oxalato monodentate complex. J Pharm Sci 92: 436-8, 2003.
12. Spingler B, Whittington Da, Lippard S J: 2.4 crystal structure of an oxaliplatin 1,2-d (GpG) intrastrand cross-link in a DNA dodecamer duplex. Inong Chem 40: 5596-602, 2001.
13. Arnould S, Hennebelle I, Canal P, Bugat R, Guichard S: Cellular determinants of oxaliplatin sensitivity in colon cancer cell lines. Eur J Cancer 39: 112-9, 2003.
14. De Vita F, Orditura M, Matano E, Bianco R, Carlomagno C, Infusino S, Damiano V, Simeone E, Diadema M R, Lieto E, Castellano P, Pepe S, De Placido S, Galizia G, Di Martino N, Ciardiello F, Catalano G, Bianco A R. A phase II study of biweekly oxaliplatin plus infusional 5-fluorouracil and folinic acid (FOLFOX-4) as first-line treatment of advanced gastric cancer patients. Br J Cancer May 9, 92 (9): 1644-1649, 2005.
15. Lersch C, Schmelz R, Eckel F, Erdmann J, Mayr M, Schulte-Frohlinde E, Quasthoff S, Grosskreutz J, Adelsberger H: Prevention of oxaliplatin-induced peripheral sensory neuropathy by carbamazepine in patients with advanced colorectal cancer. Clin Colorectal Cancer 2: 54-8, 2002.

CRF Lipoxal
200 mg/m² weekly
Name (or code) of Participant: --Psa.Ath.
Type of cancer: stomach, Stage: IV
Before Lipoxal Treatment

| BONE MARROW FUNCTION | |
|---|---|
| HEMOGLOBIN (gr/dl) | 13.0 |
| LEUKOCYTES (1,000/mm³) | 3.7 |
| PLATELETS (1,000/mm³) | 207 |
| RENAL FUNCTION | |
| BLOOD UREA (mg %) | 22 |
| CREATININE (mg %) | 0.4 |
| URIC ACID (mg %) | 4.3 |

7 Days after 4^{th} Lipoxal infusion
DATE blood was drawn for biochemical examination 2/11/2004

| | |
|---|---|
| BONE MARROW FUNCTION | |
| HEMOGLOBIN (gr/dl) | 12.3 |
| LEUKOCYTES (1,000/mm³) | 7.1 |
| PLATELETS (1,000/mm³) | 315 |
| RENAL FUNCTION | |
| BLOOD UREA (mg %) | 15 |
| CREATININE (mg %) | 0.5 |
| URIC ACID (mg %) | 4.1 |

7 Days after 9^{th} Lipoxal infusion

| | |
|---|---|
| BONE MARROW FUNCTION | |
| HEMOGLOBIN (gr/dl) | 9.8 |
| LEUKOCYTES (1,000/mm³) | 2.6 |
| PLATELETS (1,000/mm³) | 277 |
| RENAL FUNCTION | |
| BLOOD UREA (mg %) | 17 |
| CREATININE (mg %) | 0.40 |
| URIC ACID (mg %) | 3.5 |

7 Days after 12^{th} Lipoxal infusion

| | |
|---|---|
| BONE MARROW FUNCTION | |
| HEMOGLOBIN (gr/dl) | 8.6 |
| LEUKOCYTES (1,000/mm³) | 3.0 |
| PLATELETS (1,000/mm³) | 236 |
| RENAL FUNCTION | |
| BLOOD UREA (mg %) | 18 |
| CREATININE (mg %) | 0.41 |
| URIC ACID (mg %) | 3.7 |

7 Days after 16^{th} Lipoxal infusion

| | |
|---|---|
| BONE MARROW FUNCTION | |
| HEMOGLOBIN (gr/dl) | 11.1 |
| LEUKOCYTES (1,000/mm³) | 6.1 |
| PLATELETS (1,000/mm³) | 263 |
| RENAL FUNCTION | |
| BLOOD UREA (mg %) | 29 |
| CREATININE (mg %) | 0.51 |
| URIC ACID (mg %) | 4.8 |

### Example III

### Liposomal Cisplatin Combined with Gemcitabine in Pretreated Advanced Pancreatic Cancer Patients

### A Phase I-II Study

Purpose: The presently described trial is a phase I-II study based on a new liposomally encapsulated cisplatin (produced under the brand Lipoplatin® by regulon Inc. of Mountain View, Calif.). Previous preclinical and clinical data (Phase I pharmacokinetics) led to the investigation of a combined treatment modality involving Lipoplatin® and gemcitabine.

Patients and Methods: The gemcitabine dose was kept standard at 1000 mg/m2 and the lipoplatin dose was escalated from 25 mg/m2 to 125 mg/m2. The treatment was administered to advanced pretreated pancreatic cancer patients who were refractory to previous chemotherapy which included gemcitabine.

Results: Lipoplatin® at 125 mg/m2 was defined as dose limiting (DLT) toxicity and 100 mg/m2 as the maximum tolerated dose (MTD) in combination with 1000 mg/m2 of gemcitabine. Preliminary objective response rate data showed a partial response in 2/24 patients (8.3%), disease stability in 14 patients (58.3%) for a median duration of 3 months (range 2-7 months) and clinical benefit in 8 patients (33.3%).

Conclusion: Liposomally encapsulated cisplatin is a non-toxic alternative agent to bare cisplatin. In combination with gemcitabine, it has a MTD of 100 mg/m2 and shows promising efficacy in refractory pancreatic cancer.

Cisplatin, (cis-PtCl2(NH3)2) is used world-wide for the treatment of testicular and ovarian cancer as well as for bladder, head, neck, lung and gastrointestinal tumors and many others. 1-7 Although very effective against these tumors, cisplatin has been associated with severe side effects including nephrotoxicity, 8 ototoxicity, neurotoxicity, nausea and vomiting. 7-9 Carboplatin, a cisplatin analogue, is markedly less toxic to the kidneys and nervous system than cisplatin and causes less nausea and vomiting, while generally (and certainly for ovarian cancer and non-small-cell lung cancer) retaining equivalent antitumor activity. However, hematological adverse effects are more frequent with carboplatin than with cisplatin (10,11).

Gemcitabine (under the brand Gemzar®, Eli Lily, Indianapolis, Ind.), a nucleoside analogue, is administered in combination with cisplatin as first-line treatment of patients with inoperable, locally advanced (stage IIIA or IIIB) or metastatic (stage IV) non-small-cell lung cancer and as front-line treatment for patients with locally advanced (non-resectable stage III) or metastatic (stage IIIB, IV) adenocarcinoma of the pancreas. 12-14 The main adverse reaction is myelotoxicity. The advantage of using combinations of gemcitabine with platinum has been attributed to the inhibition of the DNA synthetic pathways involved in the repair of platinum-DNA adducts. Gemcitabine and cisplatin act synergistically, increasing platinum-DNA adduct formation and inducing concentration and combination-dependent changes in ribonucleotide and deoxyribonucleotide pools in ovarian cancer cell lines (15).

Previous studies on Lipoplatin® (Regulon Inc., Mountain View, Calif.) showed: a low toxicity profile, an ability to concentrate in tumors and to escape immune cells and macrophages, a slow clearance rate from the kidneys, long circulation properties in body fluids, a half-life of 36 h in the blood, and promising therapeutic efficacy. 16 In the present Phase I-II study we attempted to explore the therapeutic efficacy and toxicity profile of the lipoplatin-gemcitabine combination, given every 14 days in advanced stage pretreated pancreatic cancer patients. Our primary objectives were to determine toxicity and the maximum tolerated dose (MTD) and our secondary aims, to determine the response rate and clinical benefit.

### Patients and Methods

Patients >18 years of age with histologically or cytologically confirmed adenocarcinoma of the pancreas and bidimensionally measurable disease, who had undergone chemotherapy pretreatment and had recurrent or non responsive disease, were enrolled in the study. Other eligibility criteria included a World Health Organization (WHO) performance status (PS) of 0-2, life expectancy of at least 3 months, adequate bone marrow reserves (granulocyte count ≧1,500/dl, platelet count ≧120,000/dl) normal renal (serum creatine concentration <1.2 mg/dl) and liver function tests (total serum bilirubin concentration, <3 mg/dl, provided that serum transaminases and serum proteins were normal), normal cardiac function with no history of clinically unstable angina pectoris or myocardial infarction, or congestive heart failure within the 6 months prior, and no central nervous system involvement. Prior surgery was allowed provided that it had taken place at least 3 weeks before. Patients with active infection, malnutrition or a second primary tumor (except for a non-melanoma skin epithelioma or in situ cervix carcinoma) were excluded from the study. All patients gave their written informed consent to participate in the study.

### Treatment Plan

The plan was to combine Lipoplatin® with gemcitabine. Lipoplatin®, supplied by Regulon Inc., was administered as an 8 h i.v. infusion on days 1 and 15; 8 hours was chosen in order to be able to control possible adverse effects on the basis of our experience in the phase I trial. Gemcitabine was given as a 60 min i.v. infusion in 500 ml normal saline on days 1 and 15 at a dose of 1000 mg/m2 and cycles were repeated every 4 weeks (28 days). The infusions on days 1 and 15 were considered to be 1 cycle. Provided that patients had recovered sufficiently from the drug-related side effects, standard ondansetron antiemetic treatment was to be administered to all patients. Prophylactic administration of recombinant human granulocyte colony-stimulating factor (rhG-CSF) was not allowed. In cases of grade 3 neutropenia, these patients would receive subsequent infusions of pegfilgrastim 6 mg, on the 6th or 7th day and treatment would be postponed for one week. Treatment was administered for at least three cycles or until disease progression. The study was a phase I/II cohort, dose escalation trial of Lipoplatin® and gemcitabine. Its aims were to determine the dose limiting toxicity (DLT) of the combination and to define the maximum tolerated dose (MTD) as a recommended dose for phase II and to collect preliminary data on the efficacy of the drug in pretreated patients with pancreatic cancer. Myelotoxicity with Lipoplatin® as a single agent was considered very mild in a previous phase I study. 16 We started with a low dose of Lipoplatin®, combined with gemcitabine which is a myelotoxic agent, mainly to determine the extent of bone marrow adverse reaction. The starting dose of Lipoplatin® was 25 mg/m2 and increased by 25 mg/m2 per dose level (Table 1). The protocol was approved by the Ethical and Scientific Committee of the hospital.

Dose adjustment criteria were based on hematological parameters. In cases of grade 3 or 4 febrile neutropenia, subsequent cycles were repeated with pegfilgrastim prophylactic administration, as described above. In cases of febrile neutropenia or grade 3 or 4 neutropenia, despite the administration of rhG-CSF, gemcitabine and Lipoplatin doses were reduced by 25% in the following treatment infusion. In cases of grade 3 or 4 thrombocytopenia lasting for >5 days, the doses of both drugs were also reduced by 25%. Toxicities were graded according to WHO guidelines.

### Patient Evaluation

Pretreatment evaluation included complete medical history and physical examination, full blood cell count including differential leukocyte and platelet counts, a standard biochemical profile (and creatinine clearance when necessary), serum carcinoembryonic antigen (CEA), and CA 19-9 determinations, electrocardiogram, chest X-rays, ultrasound of the upper abdomen, and computed tomography (CT) scans of the chest, upper and lower abdomen. Additional imaging studies were performed upon clinical indication. Full blood counts with differential were performed weekly; in case of grade 3 or 4 neutropenia or grade 4 thrombocytopenia, full blood counts with differential were evaluated daily until the absolute granulocyte count was >1,000/dl and the platelet count >75,000/dl. A detailed medical and physical examination was completed before each course of treatment in order to document symptoms of the disease and treatment toxicities. Biochemical tests, ECG, serum CEA and CA 19-9 determinations, and chest X-rays were performed every 6 weeks and a neurologic evaluation was performed by clinical examination. Lesions were measured after each cycle if they were assessable by physical examination or by chest X-rays; lesions assessable by ultrasound or CT scans were evaluated after three chemotherapy cycles.

### Definition of Response

Complete response (CR) was defined as the disappearance of all measurable or evaluable disease, signs and symptoms and biochemical changes related to the tumor for at least 4 weeks, during which time no new lesions may appear. Partial response (PR) was defined as >50% reduction in the sum of the products of the perpendicular diameters of all measurable lesions compared with pretreatment measurements, lasting for at least 4 weeks, during which time no new lesions may appear and no existing lesions may enlarge. For hepatic lesions, a reduction of >30% in the sum of the measured distances from the costal margin at the midclavicular line and at the xiphoid process to the edge of the liver, was required. Stable disease (SD) was defined as <50% reduction and a <25% increase in the sum of the products of the two perpendicular diameters of all measured lesions and the appearance of no new lesions for 8 weeks. Progressive disease (PD) was defined as an increase in the product of the two perpendicular diameters of any measurable lesion by >25% over the size present at entry into the study, or, for patients who responded, the size at the time of maximum regression and the appearance of new areas of malignant disease. Bilirubin increase without recovery after endoscopic retrograde choledocho-pancreatography (ERCP) or stent set was considered as disease progression. A two-step deterioration in performance status, a >10% loss of pretreatment weight or increasing symptoms did not by themselves constitute progression of the disease; however, the appearance of these complaints was followed by a new evaluation of the extent of the disease. All responses had to be maintained for at least 4 weeks and be confirmed by an independent panel of radiologists.

### Results

### Patient Demographics

From January 2003 until December 2004, 24 patients (11 male, 13 female; median age 66 years, range 47-80 years) were enrolled in the study. The patients' characteristics are shown in Table 2. WHO performance status was 0 in 4.2% of the patients, 1 in 45.8% and 2 in 50%. The great majority of the patients were stage IV (79.2%). All patients had undergone prior chemotherapy: eleven patients with gemcitabine as a single agent treatment and 13 with gemcitabine combined with irinotecan.

### Dose Intensity

The patients received 36 courses (108 infusions every two weeks) and the median number of courses was 2 (range 1-5). Of the 24 patients, 10 patients completed 3 courses. There was no dose reduction for either drug and the patients received 99.5% of the planned dose intensity (range 93-100%) of each drug up to the fourth dosage level.

### Toxicity

No neurotoxicity or renal toxicity was observed. Temporary abdominal pain which lasted for 2-4 minutes, and which righted itself, was observed in 10/24 patients at the beginning of the Lipoplatin® infusion. Grade 3 myelotoxicity was observed in 2 out of 4 patients at the fifth dosage level. No febrile neutropenia was seen. Toxicity is shown in Tables 3 and 4. The level five dosage (125 mg/m² of lipoplatin and 1000 mg/m² of gemcitabine) was considered as DLT and dosage level 4 as the MTD. Four additional patients were treated at the fourth dosage level.

### Response to Treatment

The determination of measurable response on computed tomography was performed by two independent radiologists and two experienced oncologists. No complete responses were detected. PR was achieved in 2 patients (8.3%) with durations of 6 and 5 months. Stable disease was seen in 14 patients (58.3%) with a median duration of 3 months (range 2-7 months). Clinical benefit mainly due to pain reduction was seen in 8 patients (33.3%). At the end of the study 7 patients (29.2%) were still alive. Median survival from the beginning of second-line treatment was 4 months (range 2-8+ months).

### CONCLUSION

This new liposomally encapsulated cisplatin (Lipoplatin®) aims mainly at the avoidance of renal toxicity, which is often seen in cisplatin administration, while at the same time producing similar efficacy. The pharmacokinetics of Lipoplatin® are different from cisplatin, as has been shown in animal studies as well as in a clinical trial in patients. 16 The lack of toxicity is a major advantage, which was shown when Lipoplatin® was administered as a single agent. In the present phase I-II trial, toxicity and efficacy were studied by administering Lipoplatin® in combination with gemcitabine, an agent whose toxicity is well defined, particularly when combined with other agents. 5 The cisplatin-gemcitabine combination has been similarly used as treatment in non-small-cell lung cancer, urothelial and pancreatic cancer. 5, 7, 12 It seems that the data from the present trial indicate the advantage of very low toxicity. The every-two-week administration of the combination is very well tolerated up to the dose of 100 mg/m2 of Lipoplatin® when gemcitabine is maintained at a standard dose of 1000 mg/m2. At the dose of 125 mg/m2 of Lipoplatin®, myelotoxicity reached grades 3 and 4 and therefore this dosage was considered as DLT. The 100 mg/m2 of Lipoplatin® and 1 gr/m2 of gemcitabine were considered as the MTD. The combination achieved an objective response in 8.33% of the patients, disease stability in 58.3% and pain relief in 33.3%. Taking into account that all of the patients were refractory or in disease progression while on a prior treatment including gemcitabine, the response rate produced here should be attributed to the addition of Lipoplatin®.

Liposomally encapsulated cisplatin combined with gemcitabine administered every two weeks in advanced pretreated pancreatic cancer patients, has a MTD of 100 mg/m2 and 1000 mg/m2, respectively. It is a well tolerated treatment with promising signs of efficacy.

### REFERENCES

1. Rosenberg B: Platinum complexes for the treatment of cancer: why the search goes on, In Lippert B (ed): Cisplatin: Chemistry and Biochemistry of a Leading Anticancer Drug. Zurich, Verlag Helvetica Chimica Acta, 1999, pp 3
2. Sorenson C, Eastman A: Mechanism of cis-diamminedichloroplatinum (II)-induced cytotoxicity: role of G2 arrest and DNA double-strand breaks. Cancer Res 48: 4484-8,1988
3. Einhorn L H, Williams S D, Loehrer P J, et al: Evaluation of optimal duration of chemotherapy in favorable prognosis disseminated germ cell tumors: a Southeastern Cancer Study group protocol. J Clin Oncol 7(3): 387-91,1989
4. Aabo K, Adams M, Adnitt P, et al: Chemotherapy in advanced ovarian cancer: four systematic meta-analysis of individual patient data from 37 randomized trials. Br J Cancer 78:1479-87, 1998
5. Kaufman D, Raghavan D, Carducci M, et al: Phase II trial of gemcitabine plus cisplatin in patients with metastatic urothelial cancer. J Clin Oncol 18(9): 1921-7, 2000
6. Pignon J P, Bourhis J, Domenge C, et al: Chemotherapy added to locoregional treatment for head and neck squamous-cell carcinoma: three meta-analyses of updated individual data. Lancet 355: 949-955, 2000
7. Non-Small-Cell Lung Cancer Collaborative Group: Chemotherapy in non-small-cell lung cancer, a meta-analysis using updated data on individual patients from 52 randomized clinical trials. Br Med J 311:899-909, 1995
8. Hayes D, Cvitkovic E, Golfey R et al: High dose cis-platinum diaminedichloride: amelioration of renal toxicity by mannitol diuresis. Cancer 39(4):1372-8, 1977
9. Gandara D R, Nahhas N A, Adelson M D, et al: Randomized placebo-controlled multicenter evaluation of diethyldithiocarbamate for chemoprotection against cisplatin-induced toxicities. J Clin Oncol 13:490-496, 1995
10. Sculier J P, Lafitte J J, Lecomte J, et al: European Lung Cancer Working Party: A three-arm phase III randomized trial comparing combinations of platinum derivatives ifosfamide and/or gemcitabine in stage IV non-small-cell lung cancer. Ann Oncol 13:874-882, 2002
11. Tognoni A, Pensa F, Vaira F, et al: A dose finding study of carboplatin and gemcitabine in advanced non-small-cell lung cancer. J Chemother 14:296-300, 2002
12. Burris H A 3rd, Moore M J, Anderson J, et al: Improvements in survival and clinical benefit with gemcitabine as first-line therapy for patients with advanced pancreas cancer: A randomized trial. J Clin Oncol 15:2403-2413, 1997
13. Heinemann V, Wilke H, Mergenthaler H G, et al: Gemcitabine and cisplatin in the treatment of advanced and metastatic pancreatic cancer. Ann Oncol 11:1399-1403, 2000.
14. Stathopoulos G P, Rigatos S K, Dimopoulos M A et al: Treatment of pancreatic cancer with a combination of irinotecan (CPT-11) and gemcitabine: A multicenter phase II study of the Greek Cooperative Group for Pancreatic Cancer. Ann Oncol 14:388-394, 2003
15. Van Moorsel C J, Smid K, Voorn D A, et al: Effect of gemcitabine and cis-platinum combinations on ribonucleotide and deoxyribonucleotide pools in ovarian cancer cell lines. Int J Oncol 22:201-207, 2003
16. Stathopoulos G P, Boulikas T, Vougiouka M, et al: Pharmacokinetics and adverse reactions of a new liposomal cisplatin (Lipoplatin): Phase I study. Oncology Reports 13: 589-595, 2005.
17. Miller A B, Hoogstraten B, Staquet M, Winkler A: (WHO) Reporting results of cancer treatment. Cancer, 47(1): 207-214,1981

TABLE 1
Lipoplatin ® and Gemcitabine Dose Escalation Dose Level
No. of Patients Lipoplatin ® Gemcitabine (mg/m2 per wk)
(mg/m2 per wk) First 4 25 1000, Second 4 50 1000, Third 4 75 1000,
Fourth 4 + 4 100 1000, Fifth 4 125 1000.

| | | | Gemcitabine |
|---|---|---|---|
| Dose | No. | Lipoplatin | (mg/m² per |
| level | of patients | (mg/m² per week) | week) |
| First | 4 | 25 | 1000 |
| Second | 4 | 50 | 1000 |
| Third | 4 | 75 | 1000 |
| Fourth | 4 + 4 | 100 | 1000 |
| Fifth | 4 | 125 | 1000 |

TABLE 2
Patients' Characteristics at Baseline No % No. of patients enrolled 24
100 Age (yr) Median 66 Range 47-80 Gender Male11 45.8 Female13 54.2
Performance Status (WHO) 0 14.2 1 11 45.8 2 12 50.0 Disease Stage
III 5 20.8 IV 19 79.2 Histology Well-differentiated 3 12.5 Moderately
differentiated 12 50.0 Low differentiation 9 37.5 Previous treatment
Gemcitabine 1 gr/m2days 1, 8, 15/every 4 weeks11 45.8 Gemcitabine
900 mg/m2 + days 1, 8/every 3 weeks + 13 54.2 Irinotecan
300 mg/m2day 8/every 3 weeks

| | | No. | % |
|---|---|---|---|
| No. of patients enrolled | | 24 | 100 |
| Age (years) | | | |
| Median | 66 | | |
| Range | 47-80 | | |
| Gender | | | |
| Male | | 11 | 45.8 |
| Female | | 13 | 54.2 |
| Performance | | | |
| status (WHO) | | | |
| 0 | | 1 | 4.2 |
| 1 | | 11 | 45.8 |
| 2 | | 12 | 50.0 |
| Disease stage | | | 20.8 |
| III | | 5 | |
| IV | | 19 | 79.2 |
| Histology | | | |
| Well-differentiated | | 3 | 12.5 |
| Moderately differentiated | | 12 | 50.0 |
| Low differentiation | | 9 | 37.5 |
| Previous treatment | | | |
| Gemcitabine 1 mg/m² | Days 1, 8, 15/ | 11 | 45 8 |
| | every 4 weeks | | |
| Gemcitabine | Days 1, 8/ | 13 | 54.2 |
| 900 mg/m² + | every 3 weeks | | |
| Irinotecan | Days 8/ | | |
| 300 mg/m² | every 3 weeks | | |

TABLE 3
Hematological Toxicity by Dose Level Lipoplatin ® Gemcitabine
Toxicity Maximum Toxicity mg/m2mg/m2 No. of Pts Toxicity (grade)
Type First 25 1000-Second 50 1000-Third 75 1000-Fourth 100 1000
2/4* 2-3 Neutropenia Fifth 125 1000 2/4 3-4 Neutropenia* original
4 patients

| | | | Toxicity | Maximum | |
|---|---|---|---|---|---|
| Dose | Lipoplatin | Gemcitabine | no. of | Toxicity | Toxicity |
| level | mg/m² | mg/m² | pts | (grade) | type |
| First | 25 | 1000 | — | — | — |
| Second | 50 | 1000 | — | — | — |
| Third | 75 | 1000 | — | — | — |
| Fourth | 100 | 1000 | 2/4^{a} | 2-3 | Neutropenia |
| Fifth | 125 | 1000 | 2/4 | 3-4 | Neutropenia |

^{a}Original 4 patients

TABLE 4
Non-Hematologic Toxicity Dosage Grade 1 Grade 2 Grade 3 Grade 4
Leveln (%) n (%) n (%) n (% ) Nausea 5 (20.8)-Vomiting 2 (8.3)-
Alopecia 14 (58.3)-Fatigue 8 (33.3)-Diarrhea 2 (8.3)-
Cardiotoxicity-Neurotoxicity 3 (12.5)-Nephrotoxicity-Thrombotic
episodes 4 (16.7)-

| | Grade 1 | Grade 2 | Grade 2 | Grade 4 |
|---|---|---|---|---|
| | n (%) | n (%) | n (%) | n (%) |
| Nausca | 5 (20.8) | — | — | — |
| Vomiting | 2 (8.3) | — | — | — |
| Alopecia | 14 (58.3) | — | — | — |
| Fatigue | 8 (33.3) | — | — | — |
| Diarrhea | 2 (8.3) | — | — | — |
| Cardiotoxicity | — | — | — | — |
| Neurotoxicity | 3 (12.5) | — | — | — |
| Nephrotoxicity | — | — | — | — |
| Thrombotic | 4 (16.7) | — | — | — |
| episodes | | | | |

1. A method for forming a micelle comprising oxaliplatin, the method comprising combining an effective amount of oxaliplatin and a negatively charged phosphatidyl glycerol lipid with a solvent.

2. A method according to claim 1 wherein the solvent is ethanol and is present at 20 to 40%.

3. A method according to claim 1 wherein the negatively charged phosphatidyl glycerol lipid is dipalmitoyl phosphatidyl glycerol (DPPG), dimyristol phosphatidyl glycerol (DMPG), diaproyl phosphatidyl glycerol (DCPG), distearoyl phosphatidyl glycerol (DSPG) or dioleyl phosphatidyl glycerol (DOPG).

4. A method according to claim 3 wherein the negatively charged phosphatidyl glycerol lipid is DPPG.

5. A method according to claim 1 wherein the molar ratio of oxaliplatin to negatively charged phosphatidyl glycerol lipid is 1:1 to 2:1.

6. A method for encapsulating oxaliplatin into a liposome comprising combining an oxaliplatin micelle as defined in claim 1 with a preformed liposome or lipids.

7. A method according to claim 6 wherein the preformed liposome or lipids comprise negatively and/or positively charged lipids.

8. A method according to claim 7 wherein lipids are phospholipids or derivatives thereof.

9. A method according to claim 8 wherein the lipid is DDAB, dimethyldioctadecyl ammonium bromide; DMRIE: N-[1-(2,3-dimyristyloxy)propyl]-N,N-dimethyl-N-(2-hydroxyethyl)ammonium bromide; DMTAP: 1,2-dimyristoyl-3-trimethylammonium propane; DOGS: Dioctadecylamidoglycylspermine; DOTAP: N-(1-(2,3-dioleoyloxy)propyl)-N,N,N-trimethylammonium chloride; DOTMA: N-[1-(2,3-dioleyloxy)propyl]-n,n,n-trimethylammonium chloride; DPTAP: 1,2-dipalmitoyl-3-trimethylammonium propane or DSTAP: 1,2-disteroyl-3-trimethylammonium propane.

10. A method according to claim 7 wherein the liposome comprises one or more of cholesterol, phosphatidyl choline, phosphatdylethanolamine, hydrogenated soy phosphatidylcholine or ceraminde.

11. A method according to claim 10 wherein the preformed liposome further comprise an ammonium salt.

12. A method for encapsulating oxaliplatin into a liposome comprising the following steps:
e) forming a micelle comprising oxaliplatin by combining an effective amount of oxaliplatin and a negatively charged phosphatidyl glycerol lipid with a solvent and
f) combining said oxaliplatin micelle with a preformed liposome or lipids.

13. A method according to claim 12 wherein the solvent is ethanol and is present at 20 to 40%.

14. A method according to claim 12 wherein the negatively charged phosphatidyl glycerol lipid is DPPG, DMPG, DCPG, DSPG or DOPG.

15. A method according to claim 14 wherein the negatively charged phosphatidyl glycerol lipid is DPPG.

16. A method according to claim 12 wherein the molar ratio of oxaliplatin to negatively charged phosphatidyl glycerol lipid is 1:1 to 1:2.

17. A method according to claim 6 further comprising coating the surface of the liposome membrane with a polymer.

18. A method according to claim 17 wherein a ligand is conjugated to the polymer.

19. A method according to claim 18 wherein the ligand is capable of directing the liposome to a specific cell type with surface receptors recognized by the ligand.

20. A method according to claim 18 wherein the ligand is a peptide.

21. A method according to claim 19 wherein the ligand is selected from epidermal growth factor or an epitope thereof, endostatin, antithrombin, anastellin, angiostatin, PEX or pigment epithelial-derived factor

22. A method according to claim 1 further comprising including another antitumour drug in the micelle or liposome.

23. A method according to claim 22 wherein the drug selected from cisplatin, paclitaxel, SN-38, docetaxel, irrinotecan, 5-fluorodeoxyuridine or doxorubicin.

24. A micelle obtained by the method of claim 1.

25. A micelle comprising an effective amount of oxaliplatin and a negatively charged phosphatidyl glycerol lipid.

26. A micelle according to claim 25 wherein the phosphatidyl glycerol lipid is DPPG.

27. A micelle according to claim 25 further comprising another anticancer drug.

28. A micelle according to claim 27 wherein the drug selected from cisplatin, paclitaxel, SN-38, docetaxel, irrinotecan, 5-fluorodeoxyuridine or doxorubicin.

29. A liposome comprising oxaliplatin obtained by the method of claim 6.

30. A liposome comprising an effective amount of oxaliplatin wherein the inner and outer layer of the liposome comprise different lipids.

31. A liposome according to claim 30 comprising a negatively charged phosphatidyl glycerol lipid.

32. A micelle according to claim 31 wherein the phosphatidyl glycerol lipid is DPPG.

33. A liposome according to claim 32 further comprising one or more of cholesterol, phosphatidyl choline, phosphatdylethanolamine, hydrogenated soy phosphatidylcholine, ceramide.

34. A liposome according to claim 30 wherein the surface of the liposome is coated with a coating which allows the liposome to evade immune surveillance.

35. A liposome according to claim 34 wherein the coating is a polymer.

36. A liposome according to claim 35 wherein the polymer is PEG.

37. A liposome according to claim 35 wherein a ligand is conjugated to the polymer.

38. A liposome according to claim 37 wherein the ligand is capable of directing the liposome to a specific cell type with surface receptors recognized by the ligand.

39. A liposome according to claim 37 wherein the ligand is a peptide.

40. A liposome according to claim 37 wherein the ligand is selected from epidermal growth factor or an epitope thereof, endostatin, antithrombin, anastellin, angiostatin, PEX or pigment epithelial-derived factor.

41. A liposome according to claim 28 wherein the liposome has a particle size of 80-120 nm.

42. A liposome according to claim 28 further comprising an effective amount of another anticancer drug characterised in that oxaliplatin and the other drug are encapsulated in the same liposome.

43. A liposome according to claim 39 wherein the anticancer drug selected from cisplatin, docetaxel, paclitaxel, gemcitabine, navelbine, doxorubicin, irrinotecan, SN-38, gemcitabine or 5-fluorodeoxyuridine.

44. A liposome according to claim 28 further comprising an effective amount of an anticancer gene characterised in that oxaliplatin and the other drug are encapsulated in the same liposome.

45. A liposome according to claim 44 wherein the anticancer gene is p53, IL-2, IL-12, angiostatin, and oncostatin.

46. A liposome according to claim 27 for use as a cancer medicament.

47. The use of a liposome according to claim 27 in the manufacture of a medicament for the treatment of cancer.

48. A method of treating cancer comprising administering a liposome as defined in claim 27.

49. The use or method according to claim 46 wherein the liposome is administered weekly or biweekly by intravenous infusion 3 hour and oxaliplatin is present at a dosage of 100 to 350 mg/m².

50. The use or method according to claim 48 wherein the dosage is 100, 150, 200, 250 or 300 mg/m².

51. The use or method according to claim 49 wherein the dosage is 300 mg/m².

52. The use or method according to claim 48 wherein infusion is for 3 hours infusion once a week.

53. The use or method according to claim 48 wherein administration is in 2 to 4 cycles, each cycle lasting about 8 weeks and followed one week rest between cycles.

54. The use or method according to claim 48 wherein the cancer is selected from colorectal cancer, gastric, pancreatic, bladder, breast cancer, colorectal, gastric, oesophageal, pancreatic, urothelial, non-small cell lung, breast, prostate, head & neck, melanoma, testicular or ovarian cancer.

55. The use or method according to claim 53 wherein the cancer is colorectal, gastric or pancreatic cancer.

56. A liposome comprising an effective amount of oxaliplatin and another anticancer drug or an anticancer gene drug and a negatively charged phosphatidyl glycerol lipid.

57. A combination therapy comprising administering a liposome encapsulating effective amount of oxaliplatin and encapsulating another anticancer drug or an anticancer gene drug.

58. A liposome according to claim 55 wherein the drug selected from cisplatin, paclitaxel, SN-38, docetaxel, irrinotecan, 5-fluorodeoxyuridine or doxorubicin.

59. A combination therapy comprising administering an effective amount of gemcitabine and a liposome encapsulating an effective amount of cisplatin.

60. A combination therapy according to claim 58 wherein gemcitabine does not form part of the cisplatin liposome.

61. A combination therapy according to claim 58 wherein gemcitabine is administered at the same time as the cisplatin liposome.

62. A combination therapy according to claim 58 wherein gemcitabine is administered at a different time than the cisplatin liposome.

63. A combination therapy according to claim 58 wherein the cancer is pancreatic cancer, colorectal cancer, gastric cancer, breast cancer, non-small cell lung cancer, ovarian cancer, head and neck cancer, prostate cancer, testicular, intestinal cancer, bladder, oesophageal or urothelial cancer.

64. A combination therapy according to claim 58 wherein gemcitabine is administered at a dosage of 800 to 1000 mg/m².

65. A combination therapy according claim 64 wherein gemcitabine is administered by intravenous infusion at a dosage 1000 mg/m².

66. A combination therapy according claim 64 wherein gemcitabine is administered as a 60 min iv infusion every two weeks.

67. A combination therapy according to claim 58 wherein the cisplatin liposome is administered by intravenous infusion at a dosage of 100 to 125 mg/m².

68. A combination therapy according to claim 67 wherein the cisplatin liposome is administered as an 8 hour IV infusion every two weeks.
